(19) United States
(12) Patent Application Publication
(10) Pub. No.: US 2018/0311331 A1
(43) Pub. Date: Nov. 1, 2018

(54) PROGNOSIS AND TREATMENT OF SQUAMOUS CELL CARCINOMAS

(71) Applicants: THE REGENTS OF THE UNIVERSITY OF COLORADO, a body corporate, Denver, CO (US); SANFORD HEALTH, Sioux Falls, SD (US)

(72) Inventors: Dohun PYEON, Greenwood Village, CO (US); John LEE, Denver, CO (US)

(21) Appl. No.: 15/770,362

(22) PCT Filed: Oct. 24, 2016
(86) PCT No.: PCT/US2016/058550

§ 371 Date: Apr. 23, 2018

Related U.S. Application Data
(60) Provisional application No. 62/245,769, filed on Oct. 23, 2015.

(51) Int. Cl. A61K 39/00; A61P 35/00; C12Q 1/70; A61K 38/19; C12Q 1/6886; G01N 33/68; A61K 39/39

(52) U.S. Cl. CPC A61K 39/001136; A61P 35/00; C12Q 1/70; A61K 38/195; C12Q 1/6886; G01N 33/6863; G01N 2800/52; C12Q 2600/118; A61K 2300/00; C12Q 2600/154; C12Q 2600/158; G01N 2333/025; G01N 2333/521; A61K 39/39

(57) ABSTRACT

DNA methylation profiles predictive of head and neck squamous cell carcinoma (HNSCC) patient prognosis, as well as therapeutic protein and adoptive cell compositions useful in the treatment of HNSCC.

### GOVERNMENT INTEREST

This invention was made with Government support under grant numbers A1091968, awarded by the National Institutes of Health (NIH). The U.S. Government has certain rights in the invention.

### TECHNICAL FIELD

The invention relates to DNA methylation as a predictor of patient prognosis, specifically in the field of cancer biology, as well as therapeutic protein and adoptive cell compositions useful in the treatment of head and neck squamous cell carcinoma (HNSCC).

### BACKGROUND OF DISCLOSURE

Human papillomaviruses (HPVs) are causally associated with multiple human cancers, including cervical and head and neck cancers (HNCs) and result in about half a million deaths worldwide each year (1, 2). HPV-associated cancer progression is a multi-step process in which the cumulative effects of a number of molecular changes ultimately lead to cancer decades following initial infection. While the majority of sexually active women are infected with HPV, only about 10-20% establish persistent HPV infection and develop premalignant lesions. Among these premalignant lesions, only a small fraction will progress to invasive cancers (4).

HPV-associated oropharyngeal squamous cell carcinoma (HNSCC) incidence continues to increase dramatically and by 2020 it will likely comprise a majority of all HNSCC cases in the US and worldwide. During decades of HNSCC progression, HPV persists, evades the host surveillance, and continuously contributes to host cell proliferation and transformation. However, little is known about the molecular mechanisms of HNSCC disease progression driven by HPV, particularly in the context of host immunity.

### SUMMARY

The inventors have discovered that CXCL14 is dramatically downregulated in HPV− positive cancers. HPV suppression of CXCL14 is dependent upon HPV oncoprotein E7 and associated with hypermethylation in the CXCL14 promoter. In vivo tests revealed that murine CXCL14 re-expression clears HPV-positive tumors in immunocompetent syngeneic mice, and significantly increases CD8+ T and natural killer cell populations in tumors and tumor-draining lymph nodes.

Thus, one aspect of this disclosure is an isolated CXCL14 protein. The isolated CXCL14 protein may induce antitumor immune responses in a subject. The isolated CXCL14 protein may induce tumor clearance in vivo in a mammal. The isolated CXCL14 protein may reverse immune suppression in the tumor microenvironment by decreasing the presence and/or the effects of several chemokines, including CXCL1 and/or CXCL2. The isolated CXCL14 protein may induce in vivo clearance of an HPV-positive (HPV+) tumor in a mammal. The isolated CXCL14 protein may be an isolated CXCL14 variant that is at least 92% identical or at least 95% identical, or at least 99% identical over its entire length to a wild-type CXCL14 protein while retaining the in vivo biological activity of inducing an antitumor immune response in a subject.

The isolated CXCL14 protein may be a recombinant CXCL14 protein, including a recombinant human CXCL14 protein. The isolated CXCL14 protein may also be a modified protein, including modification such as covalent linkage to Fc protein, glycosylation, acetylation, pegylation, and/or linking to a nanoparticle, such as a metal (e.g., gold) nanoparticle. Thus, the CXCL14 protein may be provided and/or administered as a fusion protein linked to an Fc domain of IgG, and/or the heavy chain of IgG, and/or the light chain of IgG. The fusion polypeptide/protein construct of CXCL14-Fc may also be modified by acetylation or pegylation.

In another aspect, this disclosure provides a pharmaceutical composition including an isolated CXCL14 protein, including modified or fusion constructs thereof, described above, and a pharmaceutically acceptable excipient.

In another aspect, this disclosure provides a method of inducing in vivo clearance of a tumor in a subject, the method including administering any isolated CXCL14 protein, or modified version thereof, or pharmaceutical compositions described herein, to the subject in an amount sufficient to induce the clearance of the HPV+ tumor from a subject. The tumor may have at least a two-fold, or three-fold, or four-fold reduction in CXCL14 expression compared to a non-tumor tissue or a control tissue. Alternatively or additionally, the tumor may have at least a 10%, or a 20%, or a 30% or a 40%, or a 50%, or greater reduction in CXCL14 expression compared to a non-tumor tissue, or a control tissue. The tumor may be an HPV+ tumor, such as an HPV+ head and neck squamous cell carcinoma (HNSCC), cervical cancer, or anogenital cancer of the vulva, vagina, penis, or anus.

These methods of treating a patient having a tumor, may include obtaining a biological sample from the individual, analyzing the sample to determine the presence or absence of CXCL14 protein, or CXCL14 mRNA transcript levels, or CXCL14 gene hypermethylation, in the sample, and determining whether or not to administer treatment based on the presence, absence or amount of CXCL14 protein, or CXCL14 mRNA transcript levels, or CXCL14 gene hypermethylation in the sample. An isolated CXCL14 protein or pharmaceutical composition of this disclosure may be administered if CXCL14 protein activity is found to be substantially lower than wild type or a control protein activity level. An isolated CXCL14 protein or pharmaceutical composition of this disclosure may be administered if a CXCL14 mRNA transcript level in the sample is found to be substantially lower than wild type or a control CXCL14 mRNA level. An isolated CXCL14 protein or pharmaceutical composition of this disclosure may be administered if a level of CXCL14 gene hypermethylation in the sample is found to be substantially higher than wild type or a control CXCL14 gene methylation level.

The inventors have also demonstrated that Cxcl14 expression increases CD8+ T and NK cell infiltration into tumors and tumor draining lymph nodes (TDLN). They also showed that CXCL14 expressing tumor cells stimulate CD8⁺ T and NK cell migration but have little effect on macrophages and CD4+ T cells. These results show that CXCL14 plays a key role in tumor clearance by recruiting CD8+ T and NK cells into the tumor microenvironment (TME).

Thus, one aspect of this disclosure is a composition useful for adoptive cell transfer treatment of a subject having an HPV+ tumor, comprising CXCL14-induced CD8+ T and NK cells. The CXCL14-induced CD8+ T and NK cells may be produced by a method that includes immunocompatible CD8+ T and NK cells and contacting the cells with a CXCL14 protein under conditions, and for a time sufficient to generate CXCL14-induced CD8+ T and NK cells, which cells may be adoptively transferred to a subject.

A related aspect of this disclosure is a method of treating a subject having an HPV+ tumor by adoptive cell transfer of CXCL14-induced CD8+ T and NK cells to the subject. The tumor may be an HPV+ HNSCC. The method may include first analyzing a biological sample, including a tumor sample, from the subject to determine the presence or absence of CXCL14 protein activity or CXCL14 mRNA transcript or CXCL14 gene hypermethylation in the sample, and determining whether or not to administer the adoptive cell transfer treatment based on the presence, absence or amount of CXCL14 protein activity or CXCL14 mRNA transcript or CXCL14 gene hypermethylation in the sample. Adoptive cell transfer of CXCL14-induced CD8+ T and NK cells may be administered if CXCL14 protein activity is found to be substantially lower than wild type or a control protein activity level. Adoptive cell transfer of CXCL14-induced CD8+ T and NK cells may be administered if a CXCL14 mRNA transcript level in the sample is found to be substantially lower than wild type or a control CXCL14 mRNA level. Adoptive cell transfer of CXCL14-induced CD8+ T and NK cells may be administered if a level of CXCL14 gene hypermethylation in the sample is found to be substantially higher than wild type or a control CXCL14 gene CXCL14 gene hypermethylation level.

Few predictive biomarkers are available to guide patient treatment in HPV+ HNSCC beyond simple HPV testing. Currently, HPV+ HNSCC patients are treated with lower chemoradiation doses than HPV− patients.

The majority of HPV+ HNSCC patients have a better prognosis following conventional treatment (surgery and/or chemoradiation therapy) than HPV− HNSCC patients.

However, a subset of HPV+ HNSCC patients shows metastasis to locoregional lymph nodes, and nodal metastasis alone can decrease the overall survival rate of patients by nearly 50%, making status of nodal metastasis one of the most important prognostic factors in HNSCCs. Moreover, the subset of HPV+ HNSCCs with nodal metastasis has a poor prognosis with lower survival rates than HPV+ HNSCCs without nodal disease (70% vs. 93%).

The inventors have shown that there is a distinctive chemokine change during HPV-associated cancer progression with a notable decrease of CXCL14 protein and an increase of CXCL14 promoter hypermethylation. Additionally, the inventors have shown that CXCL14 promoter hypermethylation is detectable in saliva as well as tissues. And as noted earlier, CXCL14 expression clears tumor cells by increasing CD8+ T and NK cell populations in tumor and lymph nodes.

Thus, one aspect of this disclosure is the use of CXCL14 expression/promoter methylation and/or CD8+ T and NK cell infiltration as prognostic markers to determine immune responses and predict clinical outcomes in HPV+ HNSCC patients. In these methods, CXCL14 expression/promoter methylation may correlate with CD8+ T and NK cell infiltration into the tumor micro environment. In these methods, CXCL14 expression/promoter methylation may be predictive of a better clinical outcome in HPV+ HNSCC patients without nodal metastasis.

This aspect provides an in vitro method for the prognosis of HNSCC patients for progression of a cancer, including the steps of a) quantifying, in a biological sample (which may be a tumor tissue sample) from a HNSCC patient, at least one biological marker indicative of the status of the immune response of the patient against the cancer; and b) comparing the value obtained at step a) for said at least one biological marker with a predetermined reference value for the same biological marker, which predetermined reference value is correlated with a specific prognosis of progression of cancer and/or response to a specific HNSCC therapy. In these methods, step a) may include quantifying one or more biological markers selected from: the presence or absence of CXCL14 protein activity or CXCL14 mRNA transcript or CXCL14 gene hypermethylation in the biological sample. In these methods, CXCL14 expression and/or promoter methylation and/or CD8+ T and NK cell tumor infiltration are positively or negatively associated with at least one of the patient's:
i) the T stage (T1-2 vs. T3-4) and histologic grade (moderately, poorly or undifferentiated);
ii) lymph node metastasis (N0-N2a vs. N2b-N3); and,
ii) clinical outcomes (overall survival, progression-free survival, and relapse).

This disclosure also provides a kit useful for determining the presence, absence or level of CXCL14 protein activity or CXCL14 mRNA transcript or CXCL14 gene hypermethylation in a sample. The kit may include reagents useful in determining the presence or absence of CXCL14 protein activity or CXCL14 mRNA transcript or CXCL14 gene hypermethylation in a sample from a subject. The kit may include instructions for determining the ability of an individual to spontaneously clear an HPV+ tumor, including an HPV+ HNSCC. The kit may include instructions for treating an individual with an HPV+ tumor, including an HPV+ HNSCC. The kit may include instructions for treating an individual with an isolated CXCL14 protein or pharmaceutical composition of this disclosure. The kit may include instructions for treating an individual with an adoptive cell transfer composition comprising CXCL14-induced CD8+ T and NK, cells of this disclosure. The kit may also include instructions for determining the prognosis of progression of a HNSCC cancer in a patient. The kit may also include reference values or control samples useful for comparing the values for the absence or level of CXCL14 protein activity or CXCL14 mRNA transcript or CXCL14 gene hypermethylation obtained from a biological sample. The kit may also include instructions for monitoring the effectiveness of treatment (adjuvant or neo-adjuvant) of a subject with an agent by monitoring the status of the presence, absence or level of CXCL14 protein activity or CXCL14 mRNA transcript or CXCL14 gene hypermethylation in a sample from the subject over time.

This Summary is neither intended nor should it be construed as being representative of the full extent and scope of the present invention. Moreover, references made herein to “the present disclosure,” or aspects thereof, should be understood to mean certain embodiments of the present disclosure and should not necessarily be construed as limiting all embodiments to a particular description. The present disclosure is set forth in various levels of detail in this Summary as well as in the attached drawings and the Description of Embodiments and no limitation as to the scope of the present invention is intended by either the inclusion or non-inclusion of elements, components, etc. in this Summary. Additional aspects of the present disclosure will become more readily apparent from the Description of Embodiments, particularly when taken together with the drawings.

### BRIEF DESCRIPTION OF DRAWINGS

FIG. 1, shows chemokine expression changes during HPV-associated cancer progression. Gene expression levels of all known chemokines were analyzed from 128 cervical tissues in different disease stages, as indicated.

FIG. 2, shows CXCL14 expression is downregulated in CxCa and HPV+ HNSCC tissues. CXCL14 mRNA expression levels were analyzed using 128 cervical (A) and 42 HNSCC (B; HPV− HNSCC, n=26; HPV+ HNSCC, n=16) tissue samples. Normalized fluorescence intensities of gene expression are shown in box-and-whisker plots with Tukey's method for outliers (black square). P-values were determined between each transition or between HPV− and HPV+ HNCs by one-way ANOVA analysis (A) or the Student's t-test (B).

FIG. 3 shows FIG. 3. CXCL14 expression decreases in HPV+ keratinocytes. Total RNA was extracted from indicated keratinocyte lines. Expression level of CXCL14 mRNA was measured by RT-qPCR. Data are shown as relative expression (±SD) normalized by 3-actin (A-C). (D) ICC of CXCL14 was performed with NIKS and NIKS-16 cells using a CXCL14 antibody (R&D Systems).

FIG. 4 shows CXCL14 promoter hypermethylation in HPV+ cells. Genomic DNA was extracted and treated with bisulfite. MSP (A & C) and bisulfite sequencing (B) were performed as described (Song 2010). (C) MSP products of the control CXCL14 promoter and the hypermethylated CXCL14 promoter are indicated as “C” and “M”, respectively.

FIG. 5 shows that DNMT1 upregulates in HPV+ HNSCCs (A) and CxCa progression (B). DNMT1 mRNA levels were analyzed using 42 head and neck cancer (A) and 128 cervical (B) tissue samples, as described in FIG. 1. P-values were calculated between HPV− and HPV+ HNSCCs by the Student's t-test or each transition by the one-way ANOVA analysis. (C) DNMT1 mRNA expression level was measured by RT-qPCR. P-values were determined by the Student's t-test. *p<0.001, **p<0.01.

FIG. 6 shows CXCL14 re-expression by a methylation inhibitor. CaSki cells were treated with 10 μM decitabine or a vehicle control for 6 d. RT-qPCR and MSP were performed using total RNA and genomic DNA, respectively.

FIG. 7 shows Cxcl14 expression and promoter methylation in mouse oral epithelial cells. Total RNA was extracted from mouse epithelial cell lines, MOE/shPTPBL (HPV−) and MOE/E6E7 (HPV+). Cxcl14 mRNA levels were measured by RT-qPCR. P-values were calculated by the Student's t-test. p<0.0002.

FIG. 8 shows Cxcl14 expression clears HPV+ tumors in immunocompetent mice, but not in Rag1-deficient mice. Two MOE/E6E7 cell clones expressing Cxcl14 (clones 8 and 16) and one vector containing MOE/E6E7 cell clone were injected into the rear right flank of wildtype (A & B) and Rag1−/− (C & D) B6 mice (n=10, each group of wildtype; n=7, each group of Rag1−/). (A & C) Tumor growth was determined every week by the formula: volume=(width) 2×length. Survival rates were analyzed using a Kaplan-Meier estimator. (B & D) Time-to-event was determined for each group (vector only, Cxcl14-clone 8, Cxcl14-clone 16) with the event being tumor burden larger than 2,500 mm3. Deaths not associated with tumor were censored. P-values were determined by the Log-rank test.

FIG. 9 shows CXCL14 increases CD8+ T and NK cell populations in tumor and lymph nodes. MOE/E6E7 cells with Cxcl14 (clones #8 and #16) or vector were injected into the right flank of B6 mice (n=3, each group). Tumor (A) and lymph nodes (B) tissues were harvested at 21 days post injection. Percentage of immune cell populations was determined by flow cytometry.

FIG. 10 shows CXCL14 induces chemotaxis of CD8+ T and NK cells. MOE cells with Cxcl14 or vector were cultured on the bottom chamber of a Transwell. Splenocytes were added to the upper chamber. After 12 hr incubation, migrated splenocytes to the bottom chamber were collected and analyzed by flow cytometry.

FIG. 11 shows adoptive transfer of CD8+ T and NK cells induced by Cxcl14.

FIG. 12 shows workflow of the ligand-receptor complex TriCEPS technology.

FIG. 13 shows CXCR7 downregulation and promoter hypermethylation. (A) Microarray was performed as previously described. Shown are mRNA expression based on relative fluorescence intensity. (B) Genomic DNA was extracted from the same batches of NIKS cells as (A), converted by bisulfite reaction, and hybridized on the Illumina Infinium 450K methylation bead chips. Shown are the percentage changes of methylation vs. methylation in NIKS. All samples were triplicated.

FIG. 14 shows that Cxcl14 decreases myeloid-derived suppressor cells (MDSC) in Rag1−/− mice. MOE/E6E7 cell clones expressing Cxcl14 (8 and 16) and one vector containing MOE/E6E7 cell clone were injected into Rag1−/− mice (n=4, each group). Tumor (A) and spleen (B) were harvested at 23 days post injection, homogenized, and analyzed by flow cytometry. Relative abundance of MDSC cells (A & B) was determined using anti-MHCII, anti-Gr1, and anti-CD11b+ antibodies.

FIG. 15 shows that expression of CXCR2 ligands is upregulated in CxCa and HNSCC patients. CXCL1/2 and IL-8 mRNA levels were analyzed using 128 cervical and 56 head and neck tissue samples. Normalized fluorescence intensities of mRNA expression from each group are shown in box-and-whisker plots with Tukey's method for outliers (black circle). P-values were determined by one-way ANOVA analysis.

FIG. 16 shows that Cxcl14 expression decreases Treg cells. MOE/E6E7 cell clones expressing Cxcl14 (8 and 16) and one vector containing MOE/E6E7 cell clone were injected into the rear right flank of B6 mice (n=3, each group). Spleen was harvested at 21 days post injection and analyzed by flow cytometry. Relative abundance of Treg cells was determined using anti-CD4 and CD25 antibodies.

FIG. 17 shows that detection of CXCL14 promoter hyper-methylation in patient saliva samples. Genomic DNA was extracted from saliva samples and treated with bisulfite. MSP was performed as described for FIG. 4.

### DEFINITIONS

As used herein, the term “about” means +/−10% of the recited value.

As used herein, the terms isolated, purified, and the like, do not necessarily refer to the degree of purity of a cell or molecule of the present invention. Such terms instead refer to cells or molecules that have been separated from their natural milieu or from components of the environment in which they are produced. For example, a naturally occurring cell or molecule (e.g., a DNA molecule, a protein, etc.) present in a living animal, including humans, is not isolated. However, the same cell, or molecule, separated from some or all of the coexisting materials in the animal, is considered isolated. As a further example, according to the present invention, protein molecules that are present in a sample of blood obtained from an individual would be considered isolated. It should be appreciated that protein molecules obtained from such a blood sample using further purification steps would also be referred to as isolated, in accordance with the notion that isolated does not refer to the degree of purity of the cells. Moreover, an isolated CXCL14 protein of the present invention can be obtained, for example, from its natural source (e.g., human), be produced using recombinant DNA technology, or be synthesized chemically.

By “pharmaceutical composition” is meant a composition containing a CXCL14 protein or induced T cell of this disclosure, formulated with a pharmaceutically acceptable excipient, and manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other formulation described herein.

By “pharmaceutically acceptable excipient” is meant any ingredient other than the CXCL14 proteins and/or induced T cells described herein (for example, a vehicle capable of suspending or dissolving the active compound) and having the properties of being nontoxic and non-inflammatory in a patient. Excipients may include, for example: anti-adherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

The terms individual, subject, and patient are well-recognized in the art, and are herein used interchangeably to refer to a mammal, including dog, cat, rat, mouse, monkey, cow, horse, goat, sheep, pig, camel, and, most preferably, a human. The subject may be in need of anti-cancer treatment. The terms individual, subject, and patient by themselves do not denote a particular age, sex, race, and the like. Thus, individuals of any age, whether male or female, are intended to be covered by the present disclosure. Likewise, the methods of the present invention can be applied to any race, including, for example, Caucasian (white), African-American (black), Native American, Native Hawaiian, Hispanic, Latino, Asian, and European. In some embodiments of the present invention, such characteristics are significant. In such cases, the significant characteristic(s) (age, sex, race, etc.) will be indicated.

As used herein, “treatment” or “treating” is an approach for obtaining beneficial or desired results, such as clinical results. Beneficial or desired results can include, but are not limited to, alleviation or amelioration of one or more symptoms or conditions; diminishment of extent of disease, disorder, or condition; stabilization (i.e., not worsening) of a state of disease, disorder, or condition; prevention of spread of disease, disorder, or condition; delay or slowing the progress of the disease, disorder, or condition; amelioration or palliation of the disease, disorder, or condition; and remission (whether partial or total), whether detectable or undetectable. “Palliating” a disease, disorder, or condition means that the extent and/or undesirable clinical manifestations of the disease, disorder, or condition are lessened and/or time course of the progression is slowed or lengthened, as compared to the extent or time course in the absence of treatment. By “treating cancer,” “preventing cancer,” or “inhibiting cancer” is meant causing a reduction in the size of a tumor or the number of cancer cells, slowing or inhibiting an increase in the size of a tumor or cancer cell proliferation, increasing the disease-free survival time between the disappearance of a tumor or other cancer and its reappearance, preventing or reducing the likelihood of an initial or subsequent occurrence of a tumor or other cancer, or reducing an adverse symptom associated with a tumor or other cancer. In a desired embodiment, the percent of tumor or cancerous cells surviving the treatment is at least 20, 40, 60, 80, or 100% lower than the initial number of tumor or cancerous cells, as measured using any standard assay. Desirably, the decrease in the number of tumor or cancerous cells, induced by administration of a peptide or induced immune cell of this disclosure, is at least 2, 5, 10, 20, or 50-fold greater than the decrease in the number of non-tumor or non-cancerous cells. Desirably, the methods of this disclosure result in a decrease of 20, 40, 60, 80, or 100% in the size of a tumor or number of cancerous cells, as determined using standard methods. Desirably, at least 20, 40, 60, 80, 90, or 95% of the treated subjects have a complete remission in which all evidence of the tumor or cancer disappears. Desirably, the tumor or cancer does not reappear or reappears after no less than 5, 10, 15, or 20 years. By “prophylactically treating” a disease or condition (e.g., cancer) in a subject is meant reducing the risk of developing (i.e., the incidence) of or reducing the severity of the disease or condition prior to the appearance of disease symptoms. The prophylactic treatment may completely prevent or reduce appears of the disease or a symptom thereof and/or may be therapeutic in terms of a partial or complete cure for a disease and/or adverse effect attributable to the disease. Prophylactic treatment may include reducing or preventing a disease or condition (e.g., preventing cancer) from occurring in an individual who may be predisposed to the disease but has not yet been diagnosed as having the disease or disorder.

As used herein, a biological sample refers to any fluid or tissue from an individual that can be analyzed for the presence or absence of the CXCL14 protein, or the expression level or methylation state of the CXCL14 gene. Samples that can be used to practice the methods of this disclosure include, a blood sample, a saliva sample, a urine samples, a tear sample, a tissue sample, and a buccal swab. Preferred samples for extracting DNA and genotyping are blood and buccal swab samples. Methods of obtaining such samples are also known to those skilled in the art. Once a sample has been obtained, it may be analyzed to determine the presence, absence or level of CXCL14 mRNA, CXCL14 gene methylation, or CXCL14 protein. As used herein, the terms “determine,” “determine the level of CXCL14 mRNA,” “determine the amount of CXCL14 mRNA and protein,” “determine the methylation status of the CXCL14 gene”, and the like, are meant to encompass any technique which can be used to detect or measure the presence or status of CXCL14 in a sample. In this context, CXCL14 is an example of an analyte. Such techniques may give qualitative or quantitative results. CXCL14 levels can be determined by detecting the entire CXCL14 mRNA and protein or by detecting fragments, or degradation products of CXCL14.

### DESCRIPTION OF EMBODIMENTS

This disclosure provides novel methods for the prognosis of cancer in a patient, which methods are based on the detection and/or the quantification of one or more biological markers indicative of the presence of, or alternatively of the level of, the adaptive immune response of the patient against the cancer.

It has now been surprisingly shown according to this disclosure that a determination of the in situ adaptive immune response to HPV+ cancers, and especially to HPV+ HNSCCs, can be used as a parameter for predicting the clinical outcome of cancer-bearing patients.

Additionally, the surprising correlation between CXCL14 expression/promotor methylation and HPV+ tumor incidence and growth demonstrates the usefulness of methods of diagnosing squamous cell carcinomas in a subject. These methods are particularly useful in non-invasive diagnostic methods of sampling saliva samples from a subject to diagnose the presence or determine the prognosis of HNSCC tumor. The detection of hypermethylated CXCL14 DNA in such samples, including saliva samples, is particularly useful for the early detection of squamous cell carcinomas through minimally-invasive or non-invasive testing.

Further, the surprising correlation between CXCL14 expression/promotor methylation and/or CD8+ T and NK tumor cell infiltration indicates the usefulness of compositions containing, and methods of administering, CXCL14 proteins and/or CXCL14-induced CD8+ T and NK cells.

This disclosure provides pharmaceutical compositions containing isolated CXCL14 protein, or CXCL14 protein variants, useful in inducing antitumor immune responses in a subject. The isolated CXCL14 protein may induce tumor clearance in vivo in a mammal. The isolated CXCL14 protein may induce in vivo clearance of an HPV-positive (HPV+) tumor in a mammal. The isolated CXCL14 protein may be an isolated CXCL14 variant that is at least 92% identical or at least 95% identical, or at least 99% identical over its entire length to a wild-type CXCL14 protein while retaining the in vivo biological activity of inducing an antitumor immune response in a subject.

A protein variant of CXCL14 may be an isolated protein that comprises a sequence of at least 70 contiguous amino acids, wherein the at least 70 contiguous amino acid sequence is at least 92% identical, at least 94% identical, at least 96% identical or at least 98% identical over its entire length to an at least 70 contiguous amino acid sequence of the CXCL14 protein. Methods of determining the percent identity between two proteins, or nucleic acid molecules, are known to those skilled in the art.

With regard to such CXCL14 variants, any type of alteration in the amino acid sequence is permissible so long as the variant retains at least one CXCL14 protein activity described herein. Examples of such variations include, but are not limited to, amino acid deletions, amino acid insertions, amino acid substitutions and combinations thereof. For example, it is well understood by those skilled in the art that one or more amino acids can often be removed from the amino and/or carboxy terminal ends of a protein without significantly affecting the activity of that protein. Similarly, one or more amino acids can often be inserted into a protein without significantly affecting the activity of the protein.

As noted, isolated CXCL14 variant proteins may also contain amino acid substitutions as compared to the wild-type CXCL14 protein. Any amino acid substitution is permissible so long as the cytokine activity of the protein is not significantly affected. In this regard, it is appreciated in the art that amino acids can be classified into groups based on their physical properties. Examples of such groups include, but are not limited to, charged amino acids, uncharged amino acids, polar uncharged amino acids, and hydrophobic amino acids. Preferred variants that contain substitutions are those in which an amino acid is substituted with an amino acid from the same group. Such substitutions are referred to as conservative substitutions.

Naturally occurring residues may be divided into classes based on common side chain properties:

1) hydrophobic: Met, Ala, Val, Leu, Ile;

2) neutral hydrophilic: Cys, Ser, Thr;

3) acidic: Asp, Glu;

4) basic: Asn, Gin, His, Lys, Arg;

5) residues that influence chain orientation: Gly, Pro; and

6) aromatic: Trp, Tyr, Phe.

For example, non-conservative substitutions may involve the exchange of a member of one of these classes for a member from another class. In preferred embodiments, such substituted residues may be introduced into human CX protein to form an active variant useful in the therapeutic methods of this disclosure.

In making amino acid changes, the hydropathic index of amino acids may be considered. Each amino acid has been assigned a hydropathic index on the basis of its hydrophobicity and charge characteristics. The hydropathic indices are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). The importance of the hydropathic amino acid index in conferring interactive biological function on a protein is generally understood in the art (Kyte et al., 1982, J. Mol. Biol. 157:105-31). It is known that certain amino acids may be substituted for other amino acids having a similar hydropathic index or score and still retain a similar biological activity. In making changes based upon the hydropathic index, the substitution of amino acids whose hydropathic indices are within ±2 is preferred, those within ±1 are particularly preferred, and those within ±0.5 are even more particularly preferred.

It is also understood in the art that the substitution of like amino acids can be made effectively on the basis of hydrophilicity, particularly where the biologically functionally equivalent protein or peptide thereby created is intended for use in immunological embodiments, as in the present case. The greatest local average hydrophilicity of a protein, as governed by the hydrophilicity of its adjacent amino acids, correlates with its immunogenicity and antigenicity, i.e., with a biological property of the protein. The following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). In making changes based upon similar hydrophilicity values, the substitution of amino acids whose hydrophilicity values are within ±2 is preferred, those within ±1 are particularly preferred, and those within ±0.5 are even more particularly preferred. One may also identify epitopes from primary amino acid sequences on the basis of hydrophilicity.

Desired amino acid substitutions (whether conservative or non-conservative) can be determined by those skilled in the art at the time such substitutions are desired. For example, amino acid substitutions can be used to identify important residues of the CXCL14 protein, or to increase or decrease the affinity of the CXCL14 proteins described herein. Exemplary amino acid substitutions are shown in the following table:

Amino Acid Substitutions

| Original Amino Acid | Exemplary Substitutions |
|---|---|
| Ala | Val, Leu, Ile |
| Arg | Lys, Gln, Asn |
| Asn | Gln |
| Asp | Glu |
| Cys | Ser, Ala |
| Gln | Asn |
| Glu | Asp |
| Gly | Pro, Ala |
| His | Asn, Gln, Lys, Arg |
| Ile | Leu, Val, Met, Ala |
| Leu | Ile, Val, Met, Ala |
| Lys | Arg, Gln, Asn |
| Met | Leu, Phe, Ile |
| Phe | Leu, Val, Ile, Ala, Tyr |
| Pro | Ala |
| Ser | Thr, Ala, Cys |
| Thr | Ser |
| Trp | Tyr, Phe |
| Tyr | Trp, Phe, Thr, Ser |
| Val | Ile, Met, Leu, Phe, Ala |

Thus, the CXCL14 protein variants of this disclosure may comprise at least one amino acid substitution, wherein the substitution is a conservative substitution, including those substitutions shown in this table.

In another aspect, this disclosure provides a method of treating or prophylactically treating a disease or disorder in a subject, the method including administering to the subject any CXCL14 protein or CXCL14 protein variant or pharmaceutical composition containing these proteins, in an amount sufficient to treat the disease or disorder. The disease may be cancer (e.g., HNSCC) or other neoplastic diseases and associated complications. The administration of the CXCL14 protein in these treatment methods may induce the in vivo clearance of an HPV+ tumor, including an HPV+ HNSCC in a subject.

These treatment methods may include first obtaining a biological sample from the individual, and analyzing the sample to determine the presence or absence of CXCL14 protein activity or CXCL14 mRNA transcript levels or CXCL14 gene hypermethylation in the sample, and determining whether or not to administer treatment based on the presence, absence or amount of CXCL14 protein activity or CXCL14 mRNA transcript levels or CXCL14 gene hypermethylation in the sample. An isolated CXCL14 protein or CXCL14 protein variant or pharmaceutical composition of this disclosure may be administered if CXCL14 protein activity is found to be substantially lower than wild type or a control protein activity level. An isolated CXCL14 protein or pharmaceutical composition of this disclosure may be administered if a CXCL14 mRNA transcript level in the sample is found to be substantially lower than wild type or a control CXCL14 mRNA level. An isolated CXCL14 protein or pharmaceutical composition of this disclosure may be administered if a level of CXCL14 gene hypermethylation in the sample is found to be substantially higher than wild type or a control CXCL14 gene CXCL14 gene methylation level.

Due to its role in antitumor immune responses, CXCL14 is an optimal target for T-cell based therapeutic approaches including those described herein, and also including adoptive T-cell transfer. Thus, this disclosure also provides immunotherapeutic protocols involving the adoptive transfer to a subject (e.g., an HNSCC patient) of CD8+ and/or NK cells that have been induced in vitro with a CXCL14 peptide of this disclosure or that have been modified to express immunogenic CXCL14 peptides. Adoptive transfer protocols using unselected or selected T-cells are known in the art (e.g., see US patent publication Nos. 2011/0052530, and 2010/0310534; which are incorporated herein by reference) and may be modified according to the teachings herein for use with transfer cell populations containing T-cells that are specifically induced by one or more immunogenic CXCL14 peptides. Similarly, methods for transfecting/transducing T cells with desired nucleic acids have been described (e.g., see US patent publication Nos. 2004/0087025) as have adoptive transfer procedures using T-cells of desired antigen-specificity (see, e.g., Schmitt et al., 2009 Hum. Gen. 20:1240; Dossett et al., 2009 Mol. Ther. 17:742; Till et al., 2008 Blood 112:2261; Wang et al., 2007 Hum. Gene Ther. 18:712; Kuball et al., 2007 Blood 109:2331; US2011/0243972; US2011/0189141; Leen et al., 2007 Ann. Rev. Immunol. 25:243), such that adaptation of these methodologies to the methods of the present disclosure is contemplated, based on the teachings herein, including those that are directed to CXCL14 and CXCL14-derived peptides that are capable of eliciting antigen-specific T-cell responses. This disclosure therefore also includes compositions containing such CXCL14-induced T cells for administration in these methods of adoptive transfer. These compositions contain therapeutically effective amounts of the CXCL14-induced T cells and pharmaceutically acceptable excipients sufficient to maintain and administer such induced T cells to a subject in need of such administration.

Certain of the presently disclosed aspects include preventative treatment of a subject or cells, tissues or organs of a subject, that is suspected of having or of being susceptible to a condition associated with CXCL14 hypermethylation/downregulation. The preventative treatment may be the same as or different from the regimen (dosing and schedule, as well as choice of immunogenic CXCL14-derived peptide and/or other therapeutic agents such as antigen-presenting cells or adoptively transferred T-cells) employed to treat a subject or cells, tissues or organs of a subject that has been confirmed to have a cancer (such as HNSCC) associated with CXCL14 hypermethylation/downregulation.

Each publication or patent cited herein is incorporated herein by reference in its entirety.

The disclosure now being generally described will be more readily understood by reference to the following examples, which are included merely for the purposes of illustration of certain aspects of the embodiments of the present disclosure. The examples are not intended to limit the disclosure, as one of skill in the art would recognize from the above teachings and the following examples that other techniques and methods can satisfy the claims and can be employed without departing from the scope of the claimed disclosure.

### EXAMPLES

These examples demonstrate mechanisms of human papillomavirus (HPV)-induced suppression of antitumor immune responses and the development of effective prognostic and therapeutic strategies for head and neck squamous cell carcinoma (HNSCC).

The inventors' studies revealed that the chemokine CXCL14 is significantly downregulated in HPV-positive (HPV+) cancers, while many proinflammatory chemokines are upregulated. CXCL14, an approximately 9.5 kD protein constitutively secreted by skin cells, is a potential tumor suppressor, modulating immune cell recruitment and activation. Using patient tissues and cultured keratinocytes, the inventors found that CXCL14 downregulation is linked to CXCL14 promoter hypermethylation induced by the HPV oncoprotein E7. Surprisingly, restoration of CXCL14 expression in HPV+ HNSCC cells clears tumors in immunocompetent syngeneic mice, but not in Rag1-deficient mice. Mice with CXCL14 expression showed dramatically increased CD8⁺ T and natural killer (NK) cells in tumor tissues and tumor draining lymph nodes (TDLN). The inventors also found that CXCL14 induces chemotaxis of CD8⁺ T and NK cells in vitro. In contrast, myeloid-derived suppressor cells (MDSCs) are decreased in tumors and spleens of mice re-expressing CXCL14. MDSCs suppress antitumor immunity mediated by CD8⁺ T and NK cells and interfere with CD8⁺ T cell migration to the tumor. MDSCs are abundant in HNSCC patients, and correlate with severity of disease. MDSCs infiltrate into the tumor microenvironment (TME) via CXCR2 binding with IL-8, CXCL1, and CXCL2, which are highly upregulated in HNSCCs. Thus, CXCL14 downregulation by HPV drives HNSCC development by failing to elicit CD8⁺ T and NK cell responses, and by inducing MDSC infiltration into the TME, and chemokine expression and immune cell infiltration in the TME correlate to clinical outcomes of HNSCC patients.

Human papillomaviruses (HPVs) are highly prevalent pathogens that have been linked causally to 5% of all human cancers, including ˜25% of head and neck cancers (HNSCCs). Epidemiological studies have shown a 225% population-level increase in HPV-positive (HPV+) oropharyngeal squamous cell carcinoma (OPSCC) between 1988 and 2004, and HPV+ OPSCCs will likely comprise a majority of all HNSCCs in the United States by 2020. This rapid rise in prevalence of HPV+ OPSCC over the last 10 years increases the need to improve standard-of-care therapies for this particular subtype of HNSCC.

FDA-approved prophylactic HPV vaccines effectively prevent infections by several high-risk HPV types. However, these vaccines do not cover all high-risk HPVs, and their high cost greatly restricts their availability in parts of the world most in need of the vaccines. Even in the US, HPV vaccination coverage is disappointingly low, showing less than 10% among male adolescents. These vaccines also lack therapeutic effects and therefore will not impact existing HPV infections that may lead to invasive cancer decades into the future. Current studies have shown that the overall prevalence of HPV among sexually active men and women is about 50%. Therefore, there remains an urgent need to develop new tools for prognosing and treating HPV-infected individuals.

Although current therapies, including surgery, radiation therapy, and chemotherapy are effective in treating HPV+ HNSCC, patients must deal with the profound sequelae of treatment, which requires considerable support from health and social care systems. Toxicities related to current chemoradiotherapy in HNSCC can include significant local and systemic symptoms including oral mucositis, severe pain, and difficulties chewing, which often lead to dysphagia and feeding tube dependency. Fatigue, distress, disturbed sleep, and drowsiness are common additional symptoms, and symptom severity commonly increases with time in treatment. Conversely, some HPV+ HNSCCs do not respond well to therapies and progress to aggressive metastatic tumors, which are more likely to spread to multiple organs compared to HPV-negative (HPV−) HNSCCs.

A new direction of HNSCC immunotherapy based on better understanding of immune dysregulation by cancer cells will greatly reduce treatment-related morbidity. In order to develop successful patient therapies for HPV+ HNSCC, an in-depth understanding of how HPV suppresses the host immune response is mandatory. The inventors apply their understanding from animal models, patient data and tumor samples to uncover a mechanism of HPV tolerance by chemokine modulation. These studies expand our understanding of the basic mechanism of immune tolerance as well as lead to prognostic and therapeutic strategies for the management of this emerging disease.

These studies lead to a new mechanistic understanding of the roles of CXCL14 as a key communicator for local immune surveillance in oral epithelia, and uncover novel functions of antitumor immune responses through the assessment of CD8⁺ T and NK cells induced by CXCL14. Adoptive transfer of CXCL14-induced CD8⁺ T and NK cells leads to identifying therapeutic tools that can be used in the management of immunosuppressive HNSCCs. Identification of novel mechanisms by which chemokine expression in HNSCC creates the immunosuppressive TME by infiltrating MDSCs leads to discovering another effective immune checkpoint to reverse immune suppression. Assessing HNSCC clinical outcomes and patient survival associated with chemokine expression and immune cell infiltration into tumor tissues and regional lymph nodes indicates whether chemokines can be used as prognostic markers of HPV+ HNSCCs, which may aid drug design for activating this pathway to boost antitumor immune responses in HNSCC patients. Additionally, because CXCL14 is a small soluble peptide, recombinant CXCL14 may be used as a novel therapeutic drug.

Current knowledge of how viral oncogenes lead to invasion and metastasis during persistent infection is extensive. However, the virus-associated mechanisms that allow immune tolerance during cancer progression are not well understood. The inventors' previous studies have revealed that CXCL14, which induces antitumor immune responses, is suppressed by the HPV oncoprotein E7 in HPV+ HNSCC. Since CXCL14 expression is sufficient for tumor clearance in vivo, CXCL14, a ˜9.5 kD secreted protein, could be used as a drug or adjuvant in cancer immunotherapy. Additionally, tumor clearance by CXCL14 is associated with CD8⁺ T and NK cell infiltration. Thus, adoptive transfer of CXCL14-induced CD8⁺ T and NK cells may be used as another approach for cancer immunotherapy. Finally, CXCL14 expression reduces MDSC infiltration, which is known to cause the immunosuppressive TME. Thus, targeting CXCR2 that induces MDSC may be used as a target to boost antitumor immune responses in the TME.

CXCL14 expression/promoter methylation and immune cell profiles can be used as prognostic biomarkers for HNSCC patients. While HPV+ HNSCC patients show better overall prognosis, 20-30% of them show higher rates of metastasis to regional lymph nodes compared to HPV− HNSCCs. One of the biggest hurdles for prognosis of HPV+ HNSCC is the lack of useful biomarkers to measure disease stages and predict outcomes. Recent studies suggest that immune profile might be a stronger predictor of survival than TNM classification. Accordingly, there is an international effort to establish Immunoscore that enumerates antitumor immune responses. Chemokines are small secreted proteins that can be easily detected. Immune cell infiltration and immunosuppressive status have been shown to be accurate indicators and predictors of clinical outcomes. Additionally, the inventors will detect CXCL14 promoter methylation in saliva samples from HPV+ HNSCC patients, which could be a useful non-invasive method to detect the status of CXCL14. Studies to determine correlations between CXCL14 expression/promoter methylation, immune cell profiles, and clinical outcomes will develop innovative prognostic tools for HNSCC patients.

While serving as useful tools to study human cancer, xenograft models with immunodeficient mice are not feasible to study antitumor immune responses. An immunocompetent syngeneic mouse model is used to investigate antitumor immune responses with the intact immune system using genetically modifiable syngeneic HNSCC cells, providing a relevant and flexible in vivo system to study immune responses in HNSCC progression. The inventors will also use an innovative receptor-ligand capture technology, TriCEPS, to identify CXCL14 receptor(s) on CD8⁺ and NK cells. Despite recent advances of biotechnology, it is still difficult to identify cell surface receptors due to minuscule expression and insolubility. The TriCEPS technology not only can uncover CXCL14 receptors but also will serve as a powerful tool that can be broadly used in identifying receptors. Additionally, identified CXCL14 receptors will be targeted with small molecule/peptide agonists to boost antitumor immune responses in HPV+ HNSCC patients with CXCL14 loss.

The inventors' previous study demonstrated that HPV+ and HPV− HNSCCs are molecularly distinct. The cancer genome project has also documented that HPV+ HNSCCs contain far fewer mutated genes as compared to HPV− HNSCCs, suggesting that HPV plays a significant and unique role in HNSCC development.

### Example 1: Analyzing Global Gene Expression Profiles of 84 Fresh Frozen, Human Cervical and Head/Neck Tissue Specimens, Comparing HPV+ and HPV− Cancers

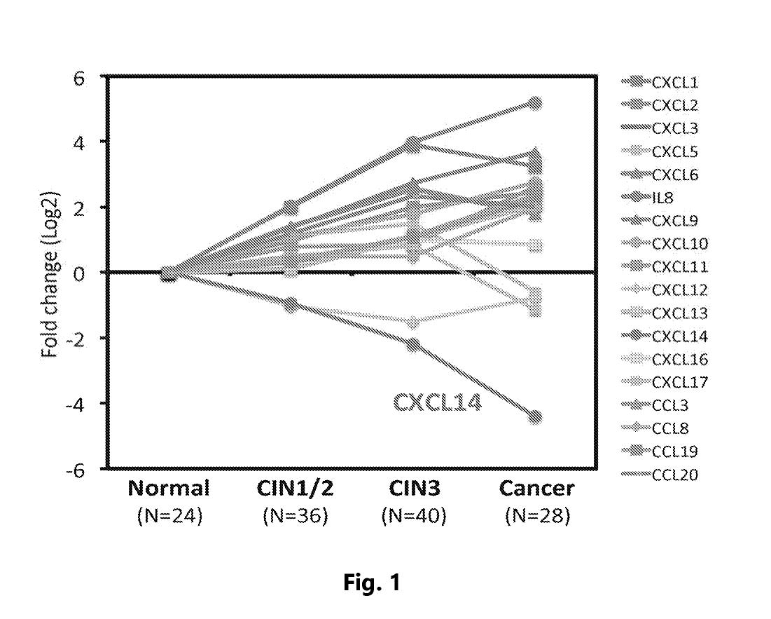

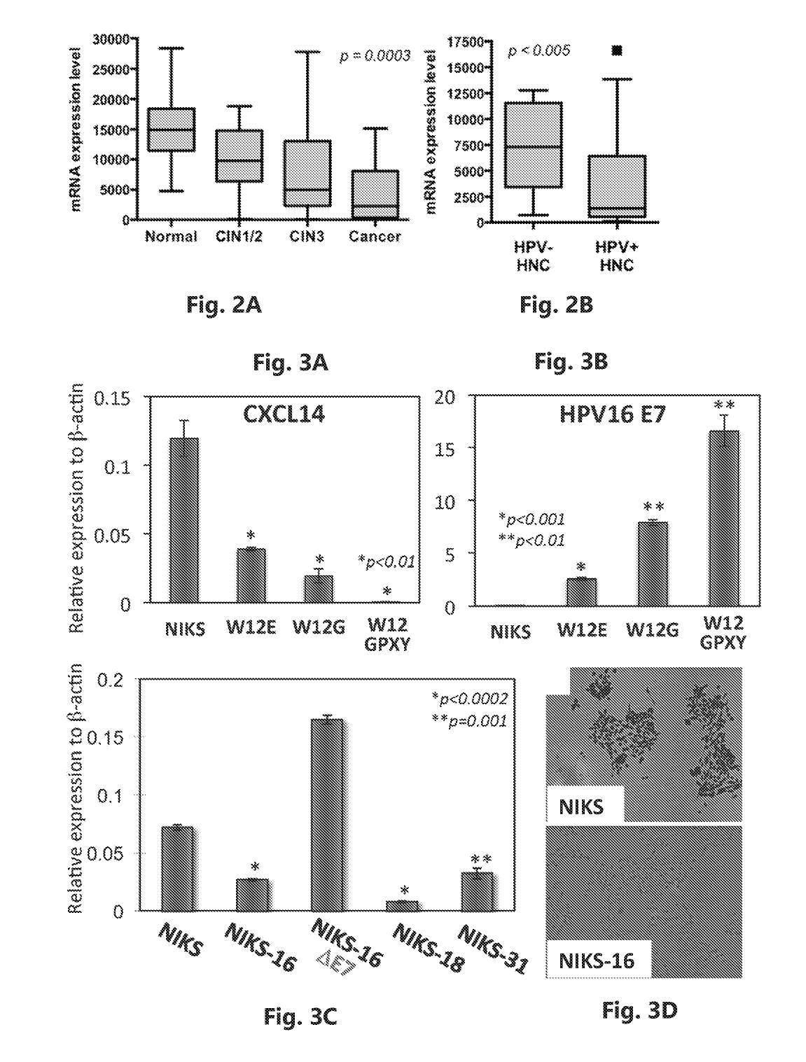

Previous results revealed striking HPV-specific gene expression signatures that allowed for distinction of HPV+ HNSCCs and cervical cancers (CxCa) from HPV− HNSCCs. These findings clearly indicate that HPV plays a pivotal role in HPV-associated cancer development. The inventors further analyzed global gene expression profiles of 128 cervical tissue specimens in different disease stages including normal, early and late premalignant epithelial lesions, and squamous cancers. The results revealed a cascade of molecular changes culminating in numerous gene expression changes at the final transition to invasive epithelial cancer. To understand immune modulation by HPV in the local microenvironment during HPV-associated cancer progression, the inventors analyzed all chemokine expression alterations using the gene expression data sets from 218 human head/neck and cervical tissue samples in different stages of cancer progression. While many proinflammatory chemokines, such as IL-8, CXCL1, and CXCL2, were highly upregulated, CXCL14 expression was dramatically downregulated in HPV+ cancers when compared to normal and HPV− HNSCC samples (FIGS. 1 and 2). CXCL14 is a relatively novel chemokine considered to be a potential tumor suppressor that modulates cell invasion/migration and host immune responses. To better understand the mechanisms by which HPV decreases CXCL14 expression, the inventors analyzed CXCL14 expression in in vitro keratinocyte culture models using reverse transcriptase-quantitative PCR (RT-qPCR). To recapitulate HPV persistent infections, the inventors used a normal immortalized keratinocyte line, NIKS and its derivatives NIKS-16, -18, and -31 containing the genome of HPV16, 18, and 31, respectively.

TABLE 1
Cell lines

| Cell type | Cell name | HPV status | Cell status |
|---|---|---|---|
| Skin | NIKS | — | Normal immortalized |
| keratinocyte | NIKS-16 | Episomal HPV16 | Normal immortalized |
| | NIKS-16ΔE7 | Episomal HPV16 | Normal immortalized |
| | | without E7 | |
| | | expression | |
| | NIKS-18 | Episomal HPV18 | Normal immortalized |
| | NIKS-31 | Episomal HPV31 | Normal immortalized |
| Cervical | W12E | Episomal HPV16 | Immortalized |
| keratinocyte | W12G | Integrated HPV17 | Immortalized |
| | W12GPXY | Integrated HPV18 | Transformed |

To mimic early, late, and cancerous cervical lesions, the inventors used the W12E cell line derived from a cervical intraepithelial neoplasia 1 (CIN1) patient, and its derivatives, W12G with integrated HPV16 genomes and a transformed cell line W12GPXY. Consistent with the results from tissue samples, CXCL14 levels continuously decreased throughout CxCa progression, showing a strong inverse correlation with HPV16 E7 expression (FIGS. 3A and 3B). In addition, CXCL14 expression is specifically downregulated in normal keratinocytes harboring high-risk HPV16, 18, or 31 genomes (FIG. 3C). Interestingly, decreased CXCL14 expression was not observed in NIKS-16ΔE7 cells containing the HPV16 genome lacking oncogene E7 expression (FIG. 3C). Using immunocytochemistry (ICC), the inventors further confirmed that CXCL14 protein expression is abrogated in NIKS-16 cells compared to HPV-keratinocytes, NIKS cells (FIG. 3D). These results show that CXCL14 downregulation is mediated by HPV oncoprotein E7.

### Example 2: CXCL14 Promoter Hypermethylation in HPV+ Cells

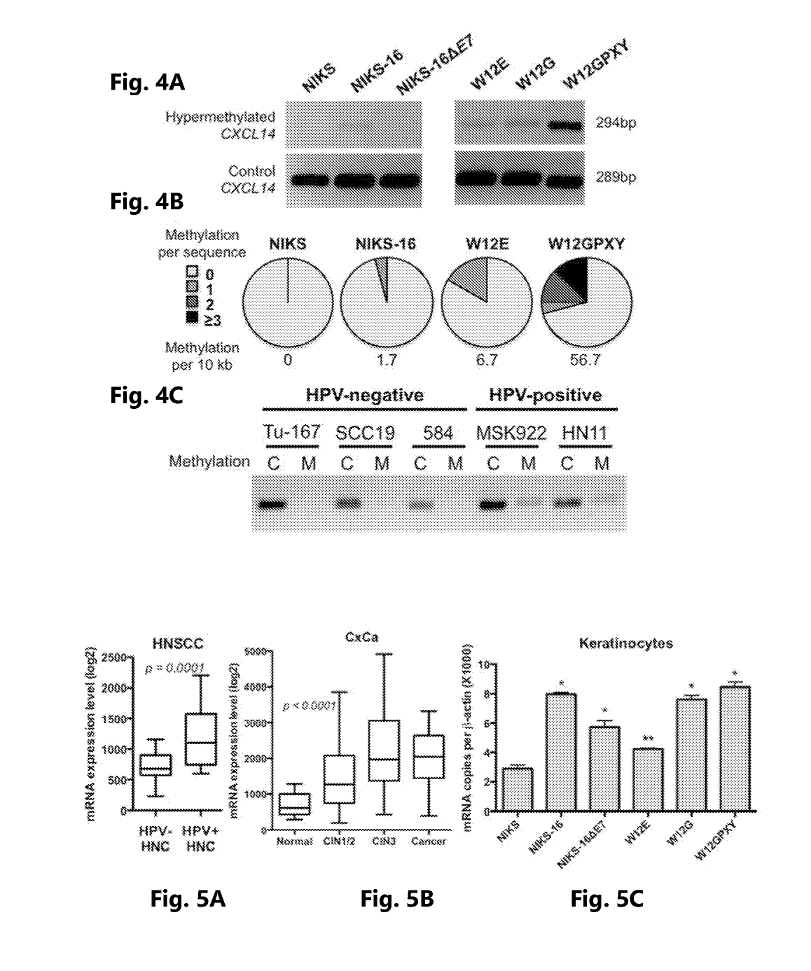

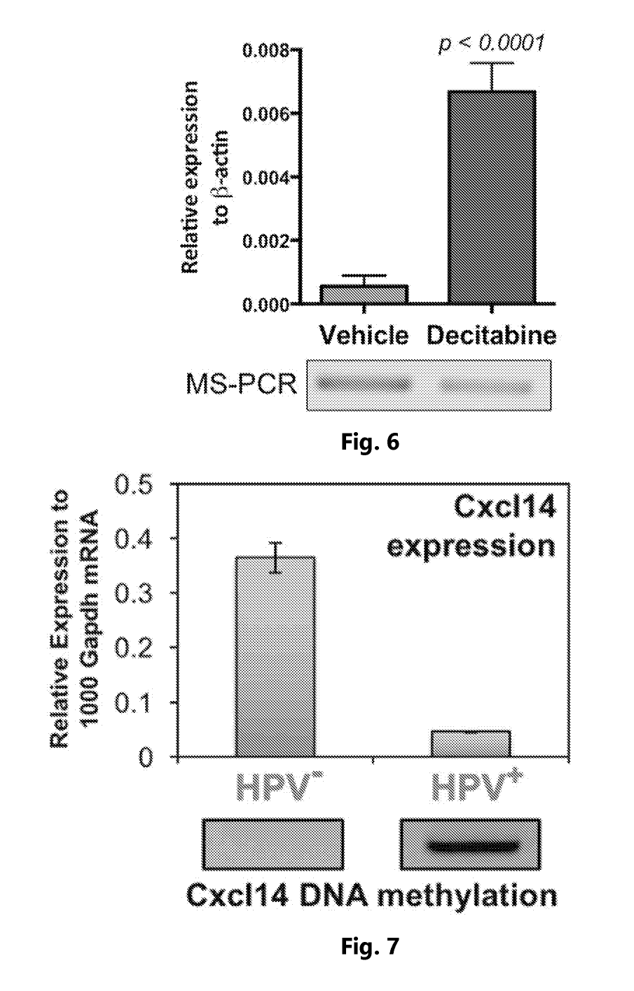

Previous studies have shown that CXCL14 expression can be suppressed by promoter hypermethylation. To determine whether decreased CXCL14 expression is linked to promoter hypermethylation, the inventors performed methylation-specific PCR (MSP) using NIKS and W12 cell lines. CXCL14 promoter methylation is inversely correlated with CXCL14 expression, and there is significantly increased CXCL14 promoter hypermethylation in HPV+ keratinocytes and HNSCC cells (FIG. 4). CXCL14 promoter hypermethylation disappears in NIKS-16ΔE7 cells. These results suggest that CXCL14 promoter hypermethylation is induced by high-risk HPVs and accumulated throughout cancer progression. A previous study showed that the HPV oncoprotein E7 activates the methyltransferase activity of DNMT1. Additionally, epigenetic silencing of many genes has been shown in HPV+ cells and in CxCa. The inventors' data also showed that DNMT1 expression is increased specifically in HPV+ HNSCC, CxCa, and NIKS-16 and W12 cells (FIG. 5A-5C). However, HPV16 E7 removal partially decreased the DNMT1 mRNA expression level (FIG. 5C). Collectively, these results show that the HPV16 oncoprotein E7 contributes to the increasing levels of DNMT1 expression during HPV-associated cancer progression. Further, treatment with the DNMT inhibitor decitabine restores CXCL14 expression in CxCa cells (FIG. 6). These results show that CXCL14 silencing by promoter methylation is mediated by the HPV oncoprotein E7.

### Example 3: CXCL14 Re-Expression in HNSCC Cells Clears Tumors Through Adaptive Immunity

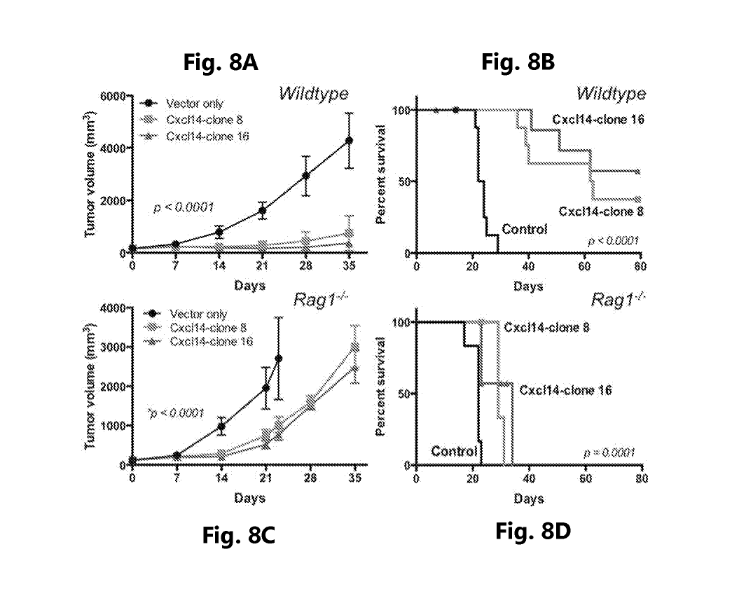

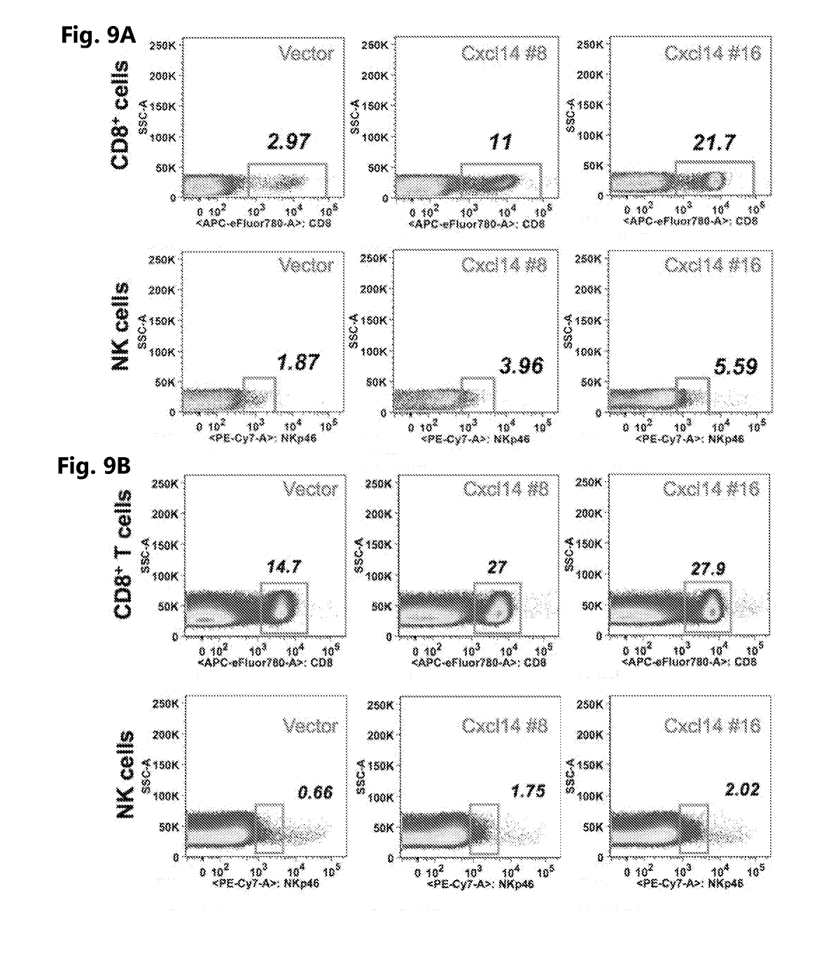

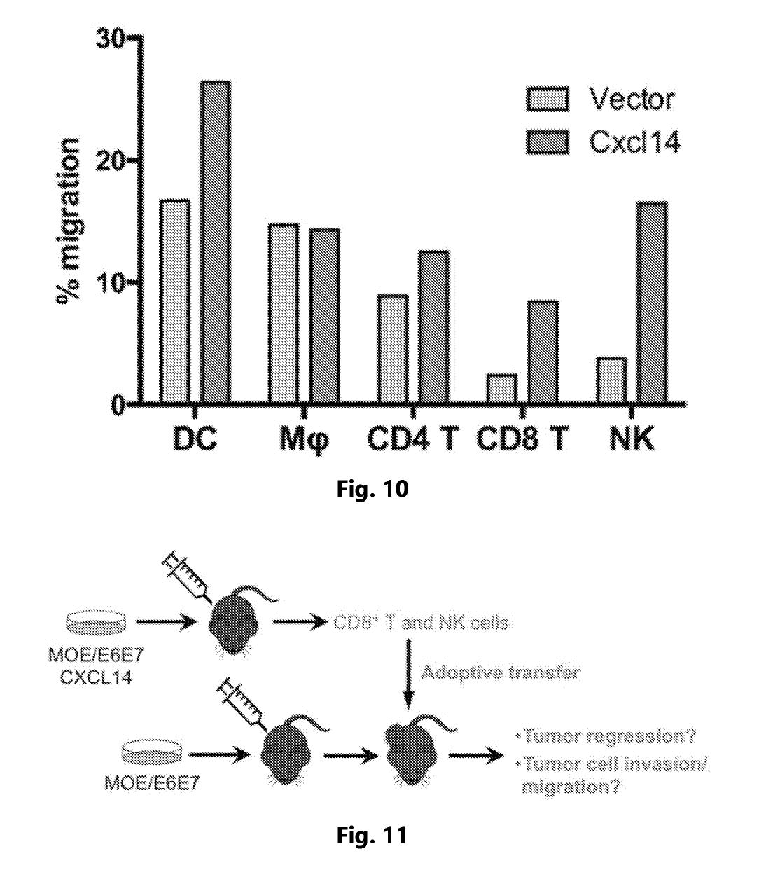

CXCL14 is an evolutionary-conserved chemokine showing 98% homology between human CXCL14 and murine Cxcl14. To determine whether CXCL14 affects tumor growth in vivo, the inventors studied mouse oropharyngeal epithelial cells (MOE/E6E7) that form tumors in immunocompetent syngeneic C57BL/6 (B6) mice. Consistent with the human cell lines and patient tissues, MOE/E6E7 cells were found to express significantly less Cxcl14 than the syngeneic HPV− MOE cells and were shown to have a highly methylated Cxcl14 promoter (FIG. 7). To test tumor suppressor functions of CXCL14, the inventors established MOE/E6E7 cell lines that re-expressed their physiological levels of Cxcl14. Strikingly, a majority of B6 mice injected with MOE/E6E7 cells expressing Cxcl14 cleared tumors, while all mice injected with control MOE/E6E7 cells succumbed to tumor burdens within 21 days (FIG. 8A). However, contrary to wildtype B6 mice, all Rag1-deficient (Rag1^{-/-}) B6 mice injected with MOE/E6E7 cells that re-expressed Cxcl14 succumbed to tumor burden within 32 days post injection (FIG. 8B). These results indicate that CXCL14-mediated tumor clearance requires adaptive immune responses. To characterize immune cell infiltration regulated by Cxcl14 expression, the inventors analyzed various immune cells in tumor tissue, tumor draining lymph node (TDLN), and in spleen harvested from the wildtype B6 mice at 21 days after injection with a control or with Cxcl14 expressing MOE/E6E7 cells. The data showed that the percentages of CD8⁺ T and NK cells were highly increased in tumor tissues and TDLNs of the wildtype B6 mice transplanted with MOE/E6E7 cells that re-expressed Cxcl14, as compared to the wildtype B6 mice injected with vector containing MOE/E6E7 cells (FIG. 9). In contrast, myeloid-derived suppressive cells (MDSCs) and regulatory T (Treg) cells were considerably decreased by Cxcl14 expression. These results suggest that CXCL14 expression is critical to the triggering of an adaptive immune response in order for CD8⁺ T and NK cells to clear transplanted HNSCC cells in vivo. To test whether CXCL14 induces direct chemotaxis of CD8⁺ T and NK cells, the inventors performed an in vitro migration assay using the Transwell system. Interestingly, migration of CD8⁺ T and NK cells to Cxcl14 expressing HNSCC cells was enhanced 3-4 fold compared to vector containing HNSCC cells (FIG. 10).

### Example 4: Determining Whether Restored CXCL14 Expression in HPV+ HNSCC Cells Promotes Antitumor CD8+ T and NK Cell Responses

Up to 90 percent of individuals infected with HPV during their lifetime will clear their HPV infection within 1-2 years without any intervention. A recent animal study showed that most immunocompetent mice are protected against mouse papillomavirus infections through CD4⁺ and CD8⁺ T cell effector functions and do not develop HPV-associated tumors. These findings suggest that host adaptive immune responses are generally effective in eliminating HPV infections and thus prevent disease progression. Recently, it has been suggested that many cancers, including HNSCCs can be cleared by intrinsic immune functions by blocking immune checkpoints such as PD-1 and CTLA-4. These results show that reversing the immune suppression in cancer patients is a promising strategy for cancer therapeutics. Previous studies have shown that the HPV16-specific CD4⁺ T cell response in CxCa patients is severely impaired and that HPV oncoprotein E7 expression in epithelium triggers immune suppression by diminishing the cytotoxic T cell response in vivo. Because these immune effector cell responses are also critical to clear tumor cells, it is likely that immune suppression triggered by HPV contributes to immune evasion of tumor cells. However, little is known about the molecular mechanisms by which HPV evades antiviral and antitumor immune responses during persistent infection and cancer progression.

The inventors have recently found that CXCL14 is dramatically downregulated in HPV+ HNSCCs as a result of E7-associated promoter hypermethylation (FIGS. 1-3). To determine whether CXCL14 affects tumor growth in vivo, the inventors utilized an HNSCC mouse model with MOE/E6E7 cells that form tumors in immunocompetent syngeneic mice. To test tumor suppressor functions of CXCL14, the inventors established MOE/E6E7 cell lines re-expressing the physiological levels of Cxcl14. Our study revealed that wild type B6 mice injected with MOE/E6E7 cells expressing Cxcl14 cleared a majority of tumors in vivo, while all mice injected with parental MOE/E6E7 cells died due to tumor burden (FIG. 8A). Additionally, Cxcl14 re-expression in MOE/E6E7 cells increases CD8⁺ T and NK cell infiltration into tumors and TDLNs (FIG. 9). Interestingly, Rag1^{-/-} mice injected with Cxcl14 expressing MOE/E6E7 cells showed delayed tumor growth. However, all mice eventually succumbed to their tumor burden (FIG. 8B). Next, the inventors determined whether CXCL14 directly induces chemotaxis of CD8⁺ T and NK cells using the Transwell system and splenocytes isolated from B6 mice. The results showed that CXCL14 expressing MOE/E6E7 cells stimulate CD8⁺ T and NK cell migration but have little effect on macrophages and CD4⁺ T cells (FIG. 10). These results suggest that CXCL14 plays a key role in the tumor clearance by recruiting CD8⁺ T and NK cells into the TME.

Previous studies have shown that NK cell activation is necessary for tumor antigen-specific CD8⁺ T cell responses in order to regress tumors. These studies also suggest that both CD8⁺ T and NK cells are important for Cxcl14-mediated HNSCC clearance. Thus, it appears that CD8⁺ and NK cells induced by CXCL14 are necessary and sufficient to clear HPV+ HNSCC.

The number of patients or experimental animals in each study was determined based on hypothesis-driven power analysis. Both numbers of patients and experimental animals will be adjusted based on the differences observed in the initial experiments.

To test whether CD8⁺ T and/or NK cells are necessary for CXCL14-mediated adaptive antitumor immune responses to clear HNSCCs, the inventors will specifically deplete CD8⁺ T and NK cells in B6 mice in vivo. Anti-mouse CD8a and anti-mouse NK1.1 antibodies will be used for CD8⁺ T and NK cell depletion, respectively. Six to eight-week old wildtype B6 mice will be intraperitoneally injected with specific antibodies. The specific cell depletion will be assessed at 24 hrs post treatment by flow cytometry using cells isolated from the spleen and lymph nodes. Control mice will be injected with isotype IgG antibodies. The mice with depleted CD8⁺ T and/or NK cells will be injected with MOE/E6E7 cells expressing CXCL14 (1×10⁵ cells) into the oral region or the right flank. Tumor volume will be measured weekly using previously established techniques. Mice will be euthanized when tumor size is greater than 1.5 cm in any dimension. Conversely, mice will be considered tumor free when no measurable tumor is detected for a period of two months. Alternatively, tumor growth and metastasis will be monitored using in vivo microCT imaging with a luciferase reporter. The inventors predict that depleting either CD8⁺ T or NK cells in mice will show tumor growth even with CXCL14 expression, similar to Rag1^{-/-} mice (FIG. 8C). NK cells have been suggested as an important link between innate and adaptive immune responses. These experiments will reveal if NK cells are necessary for CXCL14-mediated adaptive antitumor immune responses, likely by CD8⁺ T cells, to clear HNSCCs.

Although previous studies have shown effective depletion of CD8⁺ T and NK cells in mice, it is possible that a small percentage of remaining cells could show antitumor immune responses. Thus, to further examine whether CD8⁺ T and/or NK cells are necessary for CXCL14-mediated tumor suppression, the inventors will use knockout mice. MOE/E6E7 cells expressing Cxcl14 will be injected into CD8α-deficient mice and NKp46-deficient mice with the B6 background and tumor growth/metastasis and mouse survival will be monitored. Distributions of time to event outcomes (e.g. survival time) will be summarized with Kaplan-Meier curves, compared across groups using the log-rank test, and summarized using hazard ratios. ANOVA of Poisson counts will be used to compare number of nodules (metastasis) across groups. Linear mixed models will be used to describe tumor growth, and for comparisons of tumor volume at end of study. With 7 mice/group, a test of equal growth rates across groups had 80% power to detect a very small effect size of approximately 0.15 (a standardized difference in growth rates). Ten mice/group will be used to be conservative and allow for loss.

To test whether Cxcl14-induced CD8⁺ T and/or NK cells are sufficient to eliminate HNSCC in vivo, the inventors will perform adoptive transfer of the CD8⁺ T and/or NK cells harvested from mice injected with Cxcl14 expressing MOE/E6E7 cells (FIG. 11). Because adoptive transfer has been successfully used as cancer treatment, adoptive transfer may be developed as an immunotherapeutic tool to treat HNSCC patients. First, MOE/E6E7 expressing Cxcl14 will be injected into B6 donor mice and the spleen and TDLNs will be harvested at 21 days post injection. CD8⁺ T and NK cells will be isolated from splenocytes and lymphoid cells by magnetic beads using mouse CD8⁺ T Cell and NK Cell isolation kits, respectively. The isolated CD8⁺ T cells will be expanded in culture media containing IL-2 for one week. The isolated NK cells will be expanded in culture media containing IL-15 and hydrocortisone for 10 days. To track the CD8⁺ T and NK cells, the inventors will transduce the green fluorescence protein gene using lentiviruses. Tumor bearing B6 recipient mice will be prepared by injection of vector containing MOE/E6E7 cells without CXCL14 expression. Tumors of visible sizes are expected to form in 20 to 30 days post injection. CD8⁺ T or NK cells isolated from donor mice will be transferred into the tumor bearing recipient mice at 21 days post injection. For tumor bearing recipient mice, the inventors will use B6 wild type, CD8⁺ T or NK cell-depleted B6 wild type, and CD8α− or NKp46-deficient B6 mice described above. Tumor volume will be measured weekly. CD8⁺ T or NK cells isolated from mice injected with MOE/E6E7 cells containing vector will be used as controls. If Cxcl14-induced CD8⁺ T and/or NK cells are sufficient to eliminate HNSCC in vivo, the inventors will observe significant tumor suppression by adoptive transfer of CD8⁺ T and/or NK cells. Despite increased proliferation and infiltration, it is possible that CD8⁺ T or NK cells do not show effector functions. For example, the phenotype of type 1 and type 2 CD8⁺ T cells are largely different. Similarly, previous studies have shown that CD56dim NK cells are killer cells while CD56bright NK cells show more regulatory effects on NK cell effector functions. Thus, it is important to define characteristics of the CD8⁺ T or NK cells. To characterize CD8⁺ T or NK cells isolated from mice injected with MOE/E6E7 cells expressing CXCL14, the inventors will first analyze cytokine expression in the CD8⁺ T or NK cells. Using high throughput Luminex xMAP bead technology, lysates of the CD8⁺ T or NK cells will be assayed for the type 1 (IFN-γ and IL-2) and the type 2 (IL-4, IL-5, and IL-10) cytokines, as well for as other common cytokines expressed by activated CD8⁺ T and NK cells (TNF-α, RANTES, MIP-1α and MIP-1β), according to manufacturer's protocol. The multiplex and singleplex bead kits for Luminex assays will be obtained from Invitrogen and cytokine mRNA expression will be analyzed on a Luminex instrument at the UCD Cancer Center flow cytometry core facility. As negative controls, CD8⁺ T or NK cells will be also isolated from naïve B6 mice and mice injected with vector containing MOE/E6E7 cells without Cxcl14 expression. Statistical analysis will be conducted using Student's t-test using Prism software. Expression of selected cytokines will be validated using ELISA. Next, the inventors will determine the cytotoxic activity of the isolated CD8⁺ T or NK cells using the CytoTox 96 Non-Radioactive Cytotoxicity Assay (Promega). The CytoTox 96 Assay measures a stable cytosolic enzyme, lactate dehydrogenase (LDH), which is released upon cell lysis. Briefly, the isolated CD8⁺ T or NK cells will be incubated with vector containing MOE/E6E7 cells or MOE/E6E7 cells expressing Cxcl14 as target cells at variable ratios. The supernatant will be collected and cytotoxic activity will be measured using a coupled enzymatic assay. The cytotoxic activity of CD8⁺ T and NK cells against target cells will be assessed. Spontaneous LDH release will be measured by incubating target cells alone, and maximum LDH release will be determined by treating target cells with 1% Triton X-100. The inventors will also use the NK-sensitive YAC-1 and CT26 cell lines as positive controls. In case the enzymatic assay is not sensitive enough, the inventors will consider using [⁵¹Cr] chromate to label target cells and ⁵¹Cr release will be measured using a γ-counter. Based on antitumor functions of Cxcl14, the inventors predict that CD8⁺ T or NK cells isolated from mice with Cxcl14 expressing MOE/E6E7 cells will show type 1 cytokine production and significantly increased cytotoxicity. These assays can be applied to test human specimens in clinical labs.

### Example 5: Identifying CXCL14 Binding Receptors Expressed on CD8⁺ T and/or NK Cells

As a relatively new chemokine, a native receptor(s) of CXCL14 has not yet been identified. Because CXCL14 expression in HNSCC cells increases CD8⁺ T and/or NK cell infiltration into tumor in vivo (FIG. 9) and directly induces CD8⁺ T and/or NK cell chemotaxis in vitro (FIG. 10), it is very likely that both CD8⁺ T and/or NK cells express a common receptor(s) for CXCL14 signaling. While CXCL14, as a small peptide molecule, could be used as a drug as it is, identification of its receptor will expand options to develop agonists by targeting receptor. Chemokine receptors have been frequently targeted to modulate immune responses by enhancing or inhibiting their signaling. Thus, the inventors will identify CXCL14 binding receptors expressed on CD8⁺ T and/or NK cells.

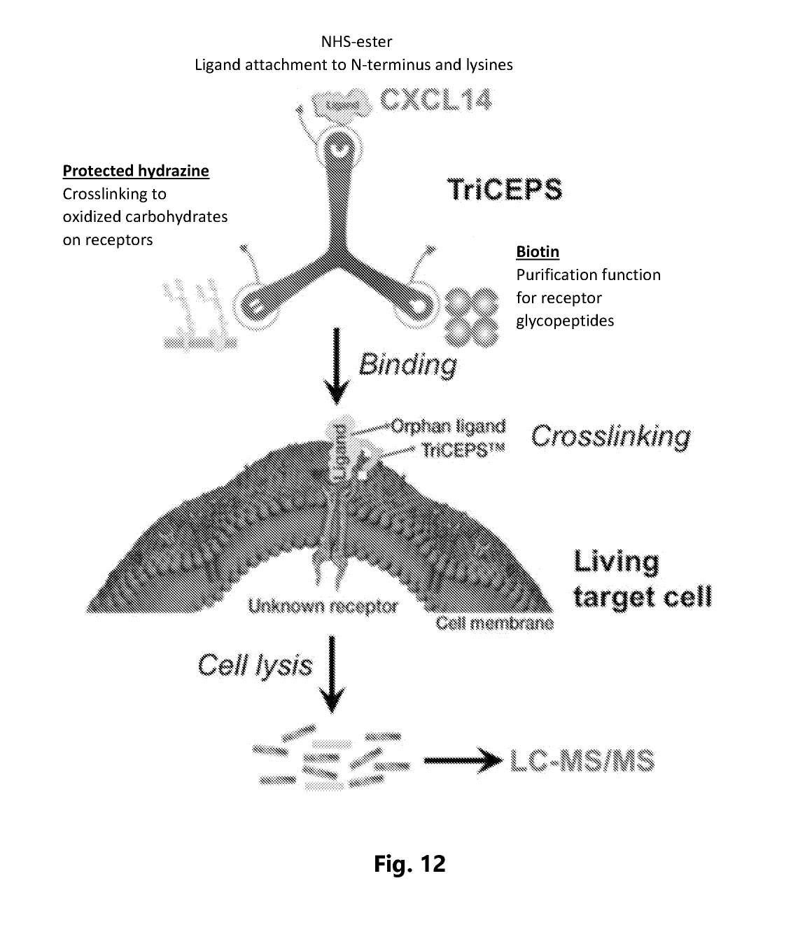

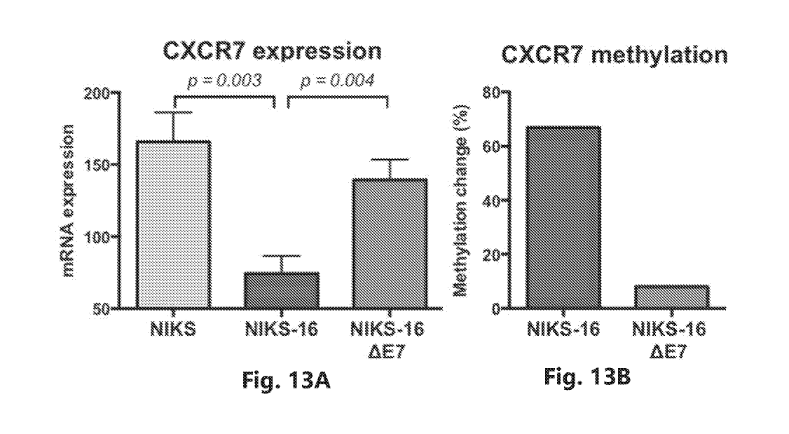

To identify a Cxcl14 receptor(s), the inventors will use a new receptor-ligand capture technology, TriCEPS. Due to minute amounts and insolubility, receptor identification is frequently unsuccessful. TriCEPS technology overcomes these obstacles by strong crosslinking between receptors and ligands. Briefly, this technology uses a chemoproteomic mediator with three arms: one arm attached to a ligand (Cxcl14 in our experiment), another arm containing protected hydrazine for crosslinking to glycosylated receptors, and a third arm with a biotin tag to purify the bound receptors. Interaction partners will be identified by liquid chromatography, followed by quantitative mass spectrometry (FIG. 12). The inventors will first produce Cxcl14 using the 293T mammalian cell system, and test activity of purified Cxcl14 using CD8⁺ T and NK cell migration in the Transwell system (FIG. 10). The inventors will couple Cxcl14 to the TriCEPS using the manufacturer's kit and validate the coupling reaction using insulin and a CD28 antibody as positive controls. CD8⁺ T and NK cells will be isolated from spleen of the mice injected with MOE/E6E7 expressing Cxcl14 and expanded. CD8⁺ T or NK cells will be incubated with the Cxcl14-conjugated TriCEPS. Crosslinking reaction will be performed with coupling buffer, and cells will be lysed by indirect sonication, and membrane proteins will be isolated and digested by trypsin. TriCEPS-captured cell surface peptides will be purified using Streptavidin Plus UltraLink Resin (Pierce). Purified peptides will be separated by reversed-phase chromatography on a high-performance liquid chromatography (HPLC) column and analyzed by mass spectrometry (MS) in our proteomics core facility. As positive controls, the inventors will use Cxcl12 and its receptor Cxcl14 that also binds to Cxcl14. As negative controls, the inventors will use macrophages and CD4⁺ T cells, which migration is not affected by Cxcl14 (FIG. 10). To validate identified Cxcl14 receptors, the inventors will perform co-immunoprecipitation using antibodies specific for identified receptors. To test whether Cxcl14 activates signaling through the identified receptors, G protein-coupled receptor (GPCR) signaling assays will be performed. Chemokine receptors contain 7-transmembrane structure for signal transduction that increases or decreases intracellular cAMP. The inventors will first prepare cell lines that stably express each identified Cxcl14 receptor. GPCR signaling activity will be measured using cAMP-Glo Assay (Promega). Downstream signaling of the Cxcl14 receptors will be further investigated using the luciferase-based GPCR Signaling 10-pathway Reporter Array (Qiagen). Using shRNA knockdown of the identified receptor, the inventors will validate the function of CXCL14-receptor interactions by testing in vivo tumor growth and in vitro cell migration. The inventors will use Spearman's correlation to assess the association between CD8⁺ T (or NK cells) and HNSCC cell. Assuming a sample size of 50, a two sided test at the 5% significance level of no association has ˜80% power to detect a moderate correlation of 0.40. Further, a 95% confidence interval will exclude zero whenever the sample correlation exceeds 0.30, and the width of these intervals is less than 0.50. The inventors may also use CXCR7, which functions in a fashion similar to CXCL14 to inhibit CXCR4 signaling. As the HPV oncoprotein E7 affects host gene expression by regulating DNA methylation, the inventors analyzed global transcriptome/methylome in human keratinocyte lines: NIKS, NIKS-16, NIKS-18, and NIKS-16ΔE7. In this analysis, the inventors found that CXCR7 expression is significantly decreased and the CXCR7 promoter is hypermethylated in NIKS-16 and NIKS-18 cells compared to NIKS and NIKS-16ΔE7 cells (FIG. 13). The inventors will test whether restoration of CXCR7 expression synergistically suppresses tumor growth using similar approaches described above. The inventors predict that one or more receptors expressed on CD8⁺ T and NK cells interacts with Cxcl4 and transduce an activation signal. If no Cxcl14 receptor is identified on CD8⁺ T and NK cells, the inventors will use the entire population of splenocytes and keratinocytes in the TriCEPS procedures. As an alternative method for receptor identification, the inventors will also consider radioisotope ([³⁵S]cysteine and [³⁵S]methionine)-based precipitation and matrix-assisted laser desorption ionization time-of-flight mass spectrometry, as previously described. Identification of CXCL14 receptor(s) on CD8⁺ T and NK cells will lead to development of useful tools to augment CXCL14 functions and thereby enhance antitumor immune responses. It will also be useful to understand signaling mechanisms by which CXCL14 boost effector functions of CD8⁺ T and NK cell to clear HNSCC cells.

### Example 6: Determining Whether Restored CXCL14 Expression in HPV+ HNSCC Cells Reverses an Immunosuppressive Microenvironment

Chronic immune suppression is required for cancer development to avoid T and NK cell effector functions that can efficiently eliminate tumor cells. During cancer progression, these effector T and NK cells are often suppressed by immune checkpoint signaling such as PD-1 and CTLA-4. In addition to suppression of effector cells through PD-1 and CTLA-4, distinct immunosuppressive cells exist in most cancer patients creating an immunosuppressive TME to evade antitumor immune responses. MDSCs are one of the major players in the immunosuppressive cellular networks of the TME. Tumor supporting MDSCs, defined as granulocytic CD11b⁺Ly6G⁺Ly6C^{low}, suppress antitumor immunity mediated by CD8⁺ T and NK cells and interfere with CD8⁺ T cell migration to tumor. The MDSC populations are abundant in the tumors, TDLNs, and peripheral blood of HNSCC patients, and correlate with disease stages. A recent phase II clinical trial has shown that a phosphodiesterase 5 (PDE5) inhibitor, tadalafil, decreases MDSCs and restores antitumor immune responses in HNSCC patients. These results strongly suggest that MDSCs are key immune cells to create an immune suppression in HNSCC patients.

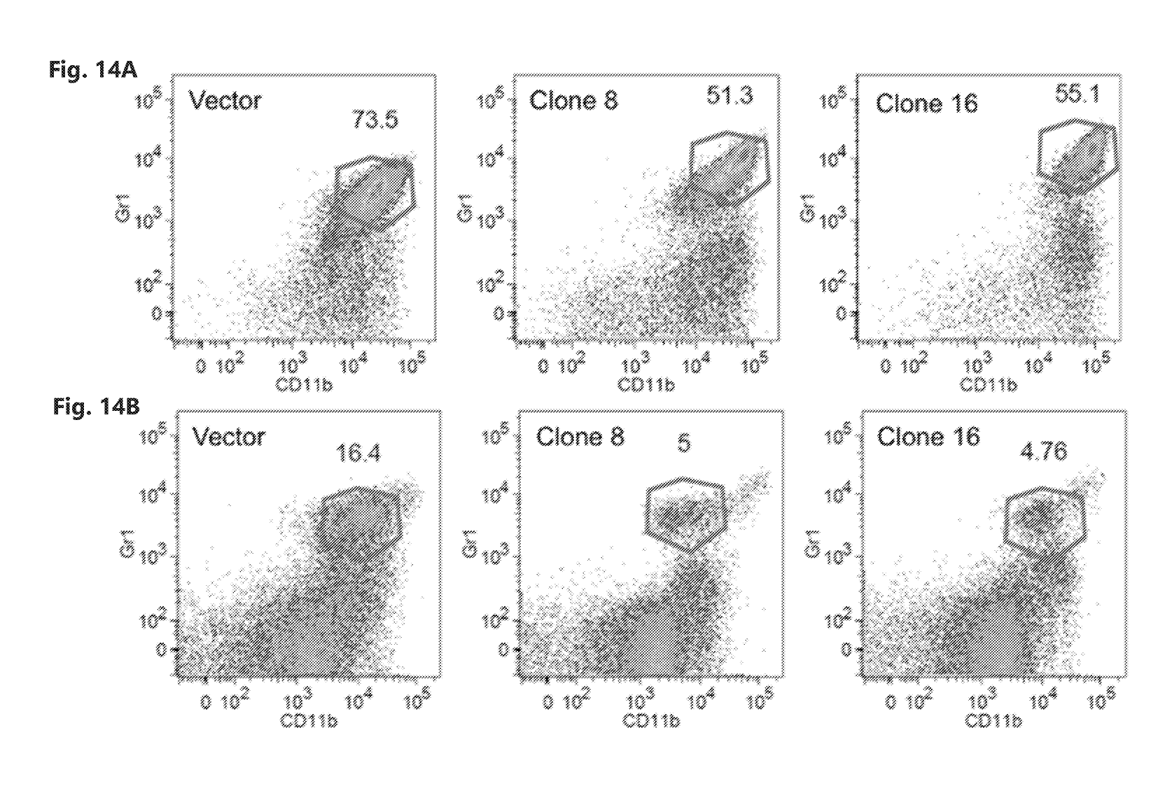

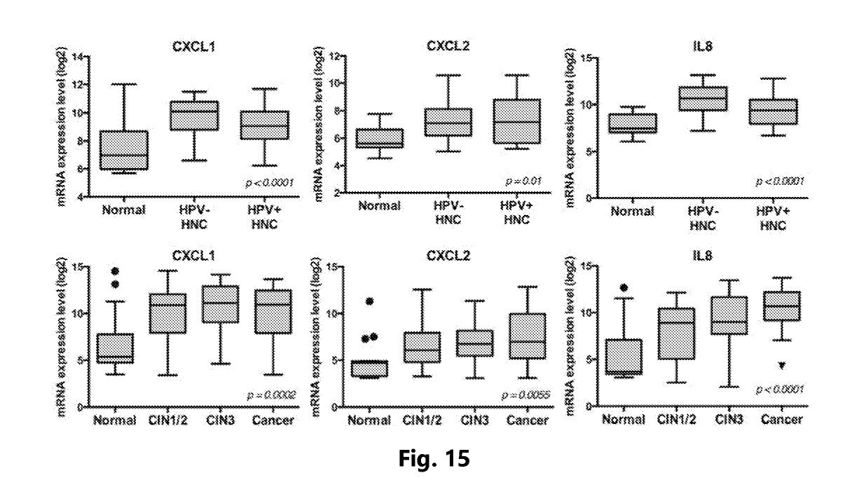

While Cxcl14 expression increases CD8⁺ T and NK cell infiltration in tumor tissue, MDSCs are significantly decreased in tumors and spleens of mice injected with MOE/E6E7 cells re-expressing Cxcl14, compared to control mice injected with cells containing an empty vector (FIG. 14). Given that MDSCs induce an immunosuppressive TME in many cancers by inhibiting CD8⁺ T and NK cells, the inventors” results indicate that the increased percentages of CD8⁺ T and NK cells in mice with Cxcl14 expression may be caused by Cxcl14-mediated MDSC reduction in the TME. The chemokine receptors CXCR1 and CXCR2 are of primary importance for the migration of granulocytes to sites of inflammation. A recent study has shown that CXCR2 is also required for MDSC infiltration into tumors for cancer development. Additionally, blocking CXCR2-mediated MDSC infiltration enhances efficacy of anti-PD1 therapy. This gene expression data show that CXCR2 ligands, IL-8, CXCL1, and CXCL2, are highly upregulated in HNSCCs (FIG. 15). These results suggest that expression of IL-8, CXCL1, and 2 from tumor cells might induce MDSC expansion and chemotaxis into the TME to create an immunosuppressive microenvironment. Another study has shown that CXCL14 directly binds to IL-8 and inhibits chemotaxis of endothelial cells. Thus, it is likely that CXCR2 ligands produced by HNSCC cells recruit MDSCs into the TME, and that CXCL14 expression interferes with IL-8 mediated MDSC infiltration. Thus, it will be verified that CXCL14 expression in HPV+ HNSCC cells reverses the immunosuppressive microenvironment by inhibiting Cxcr2-mediated MDSC expansion and infiltration. These experiments will determine novel mechanisms by which downregulation of CXCL14 triggers immune suppression in the TME.

### Example 7: Determining Whether CXCL14 Expression Reverses MDSC-Mediated Suppression of CD8⁺ T and NK Cell Responses in HNSCC

To conduct in vitro assays of CD8⁺ T and NK cell suppression by MDSCs from HPV+ HNSCC, mouse Gr-1 granulocytes containing two subsets of MDSCs, monocytic CD11b⁺Ly6G⁻Ly6C^{high} and granulocytic CD11 b⁺Ly6G⁺Ly6C^{low} MDSCs will be used. While both types of MDSCs suppress T cell proliferation and induce T cell apoptosis, granulocytic MDSCs are dominant in almost all tumors compared to monocytic MDSCs. The inventors' results also showed that granulocytic MDSCs are dramatically decreased by Cxcl14 expression (FIG. 14). To determine effects of granulocytic MDSCs on CD8⁺ T and NK cells, the inventors will perform CD8⁺ T and NK cell proliferation and apoptosis assays. Using BD FACSAria flow cytometer, granulocytic MDSCs (CD45⁺CD11b⁺Gr-1^{high}Ly6C^{low}) will be isolated from spleens of wild type B6 mice bearing HPV+ HNSCC (Stromnes 2014). CD8⁺ T and NK cells will also be purified, and labeled with carboxyfluorescein diacetate succinimidyl ester (CFSE). CD8⁺ T and NK cell proliferation will be examined in the presence or absence of the purified MDSCs. Apoptotic CD8⁺ T and NK cells will be detected using Annexin-V staining. To further investigate the effects of MDSCs on CD8⁺ T and NK cells, the inventors will measure cytokine expression levels (type 1 vs. type 2) and determine the cytotoxic activity of the isolated CD8⁺ T or NK cells. To test whether MDSCs inhibit CD8⁺ T or NK cell migration, the inventors will perform in vitro cell migration assays using a Transwell system as described in FIG. 10.

To test whether MDSCs are necessary for immune suppression in HNSCC, the inventors will specifically deplete MDSCs in B6 mice using anti-Ly6G antibodies (clone 1A8 or RB6-8C5). As an alternative to the antibody-mediated MDSC depletion, the inventors will use gemcitabine, which selectively eliminates MDSCs through apoptosis in tumor-bearing mice without any effect on B and T cells, NK cells, or macrophages. Gemcitabine treatment has shown T cell expansion and tumor regression in adoptive T cell therapy for melanoma. MDSC depletion will be verified by flow cytometry. The mice with depleted MDSCs will be injected with MOE/E6E7 cells without Cxcl14 expression and tumor growth will be monitored. If MDSC is critical for suppression of antitumor immune responses, mice with MDSC depletion will show tumor suppression similar to Cxcl14 re-expression. To examine whether MDSC depletion reverses the immunosuppressive TME, the inventors will harvest tumor tissues from the mice and profile infiltrated immune cells (T cell subsets, macrophages/DCs, neutrophils, and NK cells) using immunohistochemistry (IHC) and 12-color flow cytometry panel.

To define the mechanism by which Cxcl14 expression reduces MDSC population in the TME, the inventors will determine whether Cxcr2 signaling in MDSC is important for MDSC recruitment into the TME and suppression of CD8⁺ T and NK cells infiltration. The genes encoding IL-8 is absent in mouse and rat (Modi 1999). However, functional IL-8 homologues Cxcl1 and Cxcl2 induce chemotaxis of granulocytes including MDSCs through the interaction with the receptor Cxcr2. Both CXCL1 and CXCL2 are highly increased in human HNSCC and CxCa patient tissues (FIG. 15). To determine whether Cxcr2 is necessary for MDSC expansion and infiltration into the TME, the inventors will use Cxcr2-deficient (Cxcr2^{-/-}) mice with the B6 background. Syngeneic MOE/E6E7 cells without Cxcl14 expression will be transplanted into Cxcr2^{-/-} mice and tumor growth will be monitored comparing to tumor growth in wild type B6 mice. If Cxcr2 signaling is important for immunosuppressive functions of MDSCs, Cxcr2^{-/-} mice will show tumor clearance or delayed tumor growth without Cxcl14 expression compared to wild type mice. The inventors will also detect infiltration of MDSCs, CD8⁺ T cells, and NK cells in the TME, TDLNs, and spleen. Next the inventors will determine synergistic effects of Cxcr2 knockout and Cxcl14 expression by injecting Cxcl14 expressing MOE/E6E7 cells into Cxcr2^{-/-} mice. To test whether MDSCs are sufficient to induce immune suppression, MDSCs will be isolated from tumor-bearing wild type B6 mice and transferred into Cxcr2^{-/-} mice. If MDSCs play a key role to suppress antitumor CD8⁺ T and NK cells, adoptive transfer of MDSCs will enhance tumor growth in Cxcr2^{-/-} mice.

To determine whether Cxcl1 or Cxcl2 induce chemotaxis of MDSCs to create the immunosuppressive TME, the inventors will overexpress or knockout Cxcl1 and/or Cxcl2 in MOE/E6E7 cells. The mouse Cxcl1 or Cxcl2 genes will be delivered into Cxcl14 expressing MOE/E6E7 cells, using lentiviral transduction. Stable MOE/E6E7 cell lines expressing Cxcl1/2 and Cxcl14 will be injected into B6 mice and tumor growth will be monitored. The inventors will also detect MDSCs, CD8⁺ T cells, and NK cells in the TME and TDLNs of mice with Cxcl1/2 expressing cells using flow cytometry. If Cxcl1 or Cxcl2 induce chemotaxis of MDSCs and immune suppression, the inventors expect that tumor growth will not be suppressed even with Cxcl14 expression. Additionally, MDSCs will increase and CD8⁺ T and NK cells will decrease in the TME and TDLNs. Next, Cxcl1- or Cxcl2-deficient MOE/E6E7 cells will be established using the lentiviral CRISPR system and injected into wild type B6 mice. Tumor growth and infiltration of MDSCs, CD8⁺ T cells, and NK cells will be determined as described above. If Cxcl1 or Cxcl2 are important for MDSC infiltration and tumor growth, Cxcl1 or Cxcl2 knockout will suppress MDSC infiltration and tumor growth and increase CD8⁺ T and NK cells in the TME. If knockout of either Cxcl1 or Cxcl2 are not sufficient for tumor suppression due to a redundant function, the inventors will generate double knockout MOE/E6E7 cells of Cxcl1 and Cxcl2. To validate Cxcl14 inhibition of Cxcl1/2, the inventors will test whether MDSC migration is inhibited by Cxcl14 using the Transwell system. If Cxcl1 and Cxcl2 double knockout shows similar levels of tumor suppression to Cxcl14 expression in MOE/E6E7 cells, results suggest that Cxcr2 signaling plays a main role for inhibition of Cxcl14-mediated antitumor immune responses.

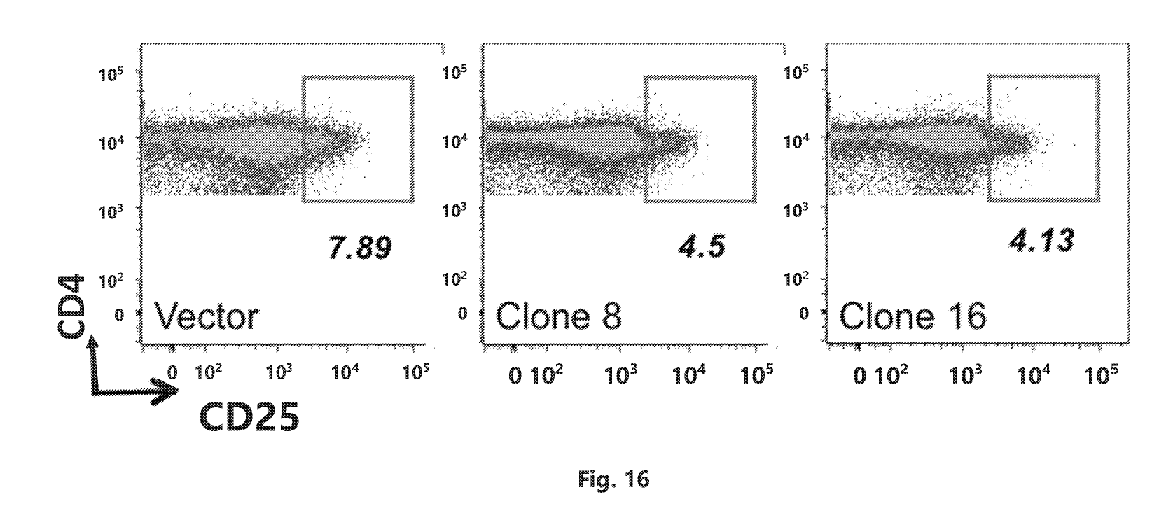

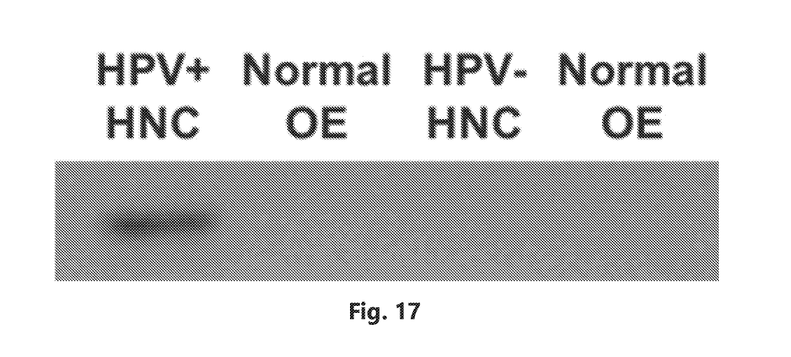

The inventors' preliminary data showed that CXCL14 re-expression in HNSCC cells significantly decrease MDSC infiltration in vivo. MDSCs, the key mediator of immune suppression, are recruited to the TME by expression of homologous proinflammatory chemokines, IL-8, CXCL1, and CXCL2 from tumor cells. Because MDSCs suppress CD8⁺ T and NK cells, these results will reveal an immunosuppressive role of MDSCs in HNSCCs, showing CXCL14-mediated increase of CD8⁺ T and NK cells and tumor suppression. But depletion of MDSCs alone may not be sufficient to reverse the immunosuppressive TME. Another immunosuppressive cell type, CD4⁺CD25⁺FoxP3 Treg cells representing a subpopulation of T cells suppress various immune cells including effector CD8⁺ T and NK cells. A recent study showed that Treg cells are increased in cetuximab-treated HNSCC patients, suppress NK cell effector functions and correlate with poor clinical outcomes. The inventors' preliminary results have also found that Treg cells are significantly decreased in spleens of mice with tumors re-expressing Cxcl14 (FIG. 16). Thus, the inventors may test whether CXCL14 expression in HPV+ HNSCC cells restores cytotoxic activity of CD8⁺ T and NK cells by suppressing Treg cell expansion.

### Example 8: Defining the Clinical Correlation Between CXCL14 Expression, Immune Cell Infiltration, and Clinical Outcomes of HNSCC Patients

Selecting patients likely to respond to a specific cancer therapy is critical for effective treatment. However, few predictive biomarkers are available to guide patient treatment in HPV+ HNSCC beyond simple HPV testing. The majority of HPV+ HNSCC patients have a better prognosis following conventional treatment (surgery and/or chemoradiation therapy) than HPV− HNSCC patients. However, a subset of HPV+ HNSCC patients shows metastasis to locoregional lymph nodes. Since nodal metastasis alone can decrease the overall survival rate of patients by nearly 50%, the status of nodal metastasis is considered one of the most important prognostic factors in HNSCCs. Moreover, the subset of HPV+ HNSCCs with nodal metastasis has a poor prognosis with lower survival rates than HPV+ HNSCCs without nodal disease (70% vs. 93%). Previous studies found that CD8⁺ T and NK cells play important roles to prevent nodal metastasis, while increased MDSCs correlate with nodal metastasis in breast cancer patients.

Recent studies have shown that immune cells and cytokines could be used as powerful prognostic biomarkers. For example, the total numbers of infiltrative CD8⁺ T and NK cells correlate with better patient survival in breast, renal, colorectal, skin, and gastric cancers. In contrast, MDSCs are regarded as a negative prognostic marker in pancreatic, esophageal, gastric, and skin cancers. It has been suggested that clinical outcomes might also be predicted by measuring expression levels of several cytokines, including IL-8 and IFN-γ in patients. These findings led to an international effort to establish Immunoscore that enumerates antitumor immune responses, which has been show as a stronger predictor of survival than TNM classification. Thus, the detection and assessment of immune cell infiltration and chemokine expression might be reliable prognostic markers that can be used in predicting clinical outcomes in HNSCC patients. Currently, HPV+ HNSCC patients are treated with lower chemoradiation doses than HPV− patients. The CXCL14^{low} HPV+ HNSCC patients may be selected and treated with higher doses and/or longer treatments/follow-ups than CXCL14^{high} HPV+ HNSCC patients.

The inventors' previous study revealed a distinctive chemokine change during HPV-associated cancer progression with a notable decrease of CXCL14 and increase of CXCL14 promoter hypermethylation (FIGS. 1, 2, and 4). CXCL14 promoter hypermethylation is detectable in saliva (FIG. 17) as well as tissues (Table 2).

TABLE 2
CXCL14 promoter methylation in HPV+ HNSCCs. CXCL14 promoter
methylation was determined by MSP using genomic DNA from 20
HPV− and 16 HPV+ HNSCC tissue samples. The levels of
hypermethylation were scored based on band density of MSP products.

Methylation status

| | − | + | ++ | +++ |
|---|---|---|---|---|
| HPV⁻ HNSCC (n = 20) | 12 | 5 | 3 | 0 |
| HPV⁺ HNSCC (n = 16) | 6 | 3 | 5 | 2 |

Given that CXCL14 expression clears tumor cells by increasing CD8⁺ T and NK cell populations in tumor and lymph nodes (FIGS. 8 and 9), CXCL14 and immune cells may be used as reliable prognostic markers to determine immune responses and predict clinical outcomes in HPV+ HNSCC patients. Thus, the inventors will show that CXCL14 expression/promoter methylation correlates with CD8⁺ T and NK cell infiltration into the TME and is predictive of a better clinical outcome in HPV+ HNSCC patients without nodal metastasis. The inventors will also establish a correlation of HNSCC clinical outcomes with CXCL14 expression/promoter methylation and immune cell infiltration into tumor tissues and regional lymph nodes. Large prospective cancer screening studies in the form of randomized clinical trials will follow in order to evaluate the clinical efficacy, the benefits and any potential harm that might ensue when CXCL14 and immune cells are used as a basis for prognosis of HPV+ HNSCCs. Approximately 1,000 HNSCC patient tissue samples have been collected (along with adjacent normal tissues and associated patient demographics, HPV status, and clinical outcomes). Among these samples, about 250 samples are HPV+. To determine if CXCL14 levels correlate to the numbers of CD8⁺ T and NK cells infiltrating the TME, the inventors will assay tissue, lymph node, saliva and blood samples from 200 HPV+ HNSCC patients. Histologically, normal distant mucosal tissue in this patient group as well as 30 normal subjects (i.e. tonsillectomy) will be used as controls. The inventors will also include 50 HPV− HNSCC patients as a comparing group. Patients with current or previous smoking history will be stratified by pack-years and duration. Therefore, HPV+ HNSCC smokers will be included as a subset group of patients.

The HPV status of each sample will be confirmed by p16 IHC and HPV PCR (14 high-risk: 16, 18, 31, 33, 35, 39, 45, 51, 52, 56, 58, 59, 66, and 68). The inventors will first evaluate CXCL14 expression/promoter methylation levels and CD8⁺ T and NK cell infiltration in these tissue samples. CD8⁺ T and NK cell populations will be determined in both tissues and surgically dissected regional lymph nodes or in fine needle aspirate (FNA) nodal samples for patients who will receive chemoradiation as definitive treatment. Next, the inventors will determine whether CXCL14 expression/promoter methylation and CD8⁺ T and NK cell infiltration are positively or negatively associated with: i) the T stage (T1-2 vs. T3-4) and histologic grade (moderately, poorly or undifferentiated); ii) lymph node metastasis (N0-N2a vs. N2b-N3) and iii) clinical outcomes (overall survival, progression-free survival, and relapse). The inventors will assess for gender differences in CXCL14 expression/promoter methylation and CD8⁺ T and NK cell infiltration in this patient population. Based on a previous study, 3-year overall and 3-year progression-free survival rates of HPV+ HNSCC patients are ˜80% and ˜70%, respectively. About 40% of HPV+ HNSCCs show N2b-N3 stages of lymph node metastasis at time of diagnosis. Patient will be clinically followed for 5 years with surveillance examination and scheduled imaging (PET-CT, CT, MRI) to assess for locoregional relapse as well as distant metastasis. Those with recurrence or metastasis during this initial period will be followed until 5-year disease-free interval has been achieved. Results will be validated with samples from 100 prospective HPV+ HNSCC patients.

The inventors will analyze mRNA levels of CXCL14 in HNSCC tissue samples using our RT-qPCR procedures (FIG. 3A-3C). Scrape-prepared (macrodissected) epithelial tissues from pre-mapped tissue sections will be provided. Laser capture microdissection will be performed if the tissue contains less than 80% of normal epithelial or tumor cells based on the assessment of prior H&E stained tissue. Next, the inventors will measure CXCL14 protein levels using IHC with anti-CXCL14 antibodies. Preliminary immunostaining has shown a clear difference between CXCL14 protein expression in NIKS (HPV−) and NIKS-16 (HPV+) cells (FIG. 3D). For future tests in a CLIA-certified laboratory, the inventors will begin with strict plans for positive and negative controls. Using western blotting, a single approximately 10 kD band for CXCL14 expression will be detected in 10 CXCL14-positive normal tissue samples and 10 CXCL14-negative HNSCC tissue samples for positive and negative controls, respectively. The inventors will also analyze CXCL14 promoter methylation in tissue and saliva samples using methylation-specific PCR (MSP). For a standard test in a CLIA-certified laboratory, HPV+ HNSCC cell lines (MSK922 and HN11) and normal oral keratinocytes will be used as positive and negative controls, respectively. The preliminary data showed that high levels of CXCL14 methylation are more frequently detected in HPV+ HNSCCs than HPV− HNSCCs (Table 2). However, 40% of HPV− HNSCCs also has methylated CXCL14. Thus, the inventors will determine whether CXCL14 methylation status in HPV− HNSCC correlates to clinical outcomes as well as in HPV+ HNSCCs.

Total numbers of CD8⁺ T and NK cells infiltrated into tumor and lymph node will be assessed using IHC with anti-human CD8α and NKp46 antibodies, respectively. Additionally, the inventors will detect immunosuppressive cells including MDSCs and Treg cells, which the inventors have observed to be decreased in Cxcl14 expressing mice (FIGS. 14 and 16). To detect MDSCs and Treg cells, the inventors will perform double IHC labeling as described. All IHC and image analysis will be performed. The IHC images will be imported using an Aperio scanner and analyzed using NIH Image J. The number of positive cells will be quantified automatically, according to established assessment criteria. The inventors will also analyze CD8⁺ T, NK, MDSC, and Treg cells in blood and/or nodal samples of the patients using multi-color flow cytometry. Peripheral mononuclear cells (PBMCs) will be isolated from patient blood samples, and analyzed using multicolor flow cytometry with our panel of antibodies conjugated with unique fluorophores: neutrophils (Gr1^{high}), DCs (MHCII⁺, CD11c⁺), macrophages (MHCII⁺, F4/80⁺), monocytes (Gr1^{mid}), CD4⁺ T cells (CD4⁺), CD8⁺ T cells (CD8⁺), Treg cells (CD4⁺, CD25⁺), NK cells (NKp46⁺), and MDSCs (MHCII^{low}, Gr1⁺, CD11b⁺) (manuscript in revision). For positive and negative controls to validate infiltrated immune cells in a CLIA-certified laboratory, the inventors will isolate CD8⁺ T cells, NK cells, and MDSCs from PBMCs using magnetic bead selection and detect specific markers using western blot. Quantification of immunostaining will be performed.

To assess whether biomarkers, primarily CXCL14 levels and CD8⁺ T and NK cell numbers, correlate with survival, relapse and lymph node metastasis, a first step will assess all pairwise associations among biomarkers and patient clinical/demographic characteristics, without adjustment for multiple comparisons. Cox proportional hazards and logistic regression models will then be used to assess associations of individual biomarkers with time time-to-event (OS, time to relapse), and binary (i.e. metastasis or relapse Y/N) outcomes, adjusting for confounders among patient characteristics. These analyses will inform the final step, where the Akaike information criterion (AIC) will be used to build multivariable Cox and logistic prediction models that consider all biomarkers as potential predictors, and adjust for confounders. A test in the Cox model at the 5% significance level has 80% power to detect an adjusted hazard ratio of 1.60 for a one SD increase on a continuous predictor (CXCL14 expression level or an immune cell number), and an adjusted hazard ratio of 2.54 for a binary predictor with a 50-50 split, both assuming an R-squared of 0.10 between the marker and other predictors in the model and a predicted 20% rate of progression or death over the study. The minimum detectable hazard ratio is smaller is event rate is larger (0.3). Similarly, a test in a logistic regression model at the 5% level has 80% power to detect an adjusted odds ratio of 1.69 for a one SD increase in a continuous predictor and an adjusted odds ratio of 2.55 for a binary variable with a 50-50 split, both assuming an R-squared of 0.10 between the predictor and the other predictors in the model. The baseline rate is assumed to be 0.20. All power calculations were based on a sample size of 200.

Because the inventors' studies have shown that immune responses in the TME are critical for tumor clearance, the inventors predict that CXCL14 levels and CD8⁺ T and NK cell numbers closely correlate and CXCL14 promoter methylation status inversely correlates to higher survival rates and lower rates of relapse and nodal metastasis. But CXCL14 levels may be variable and not sufficient to reach significant correlations with clinical outcomes. In this case, the inventors will further analyze expression of proinflammatory chemokines (IL-8, CXCL1, CXCL2). Further, analysis of type 1 (IFN-γ, IL-12p70) vs. type 2 (IL-4, IL-6, IL-10) cytokines in blood will be considered to determine systemic changes of immune responses and its correlations with clinical outcomes of HNSCC patients. Prognosis of HPV+ HNSCC may be good in short-term but long-term the patients may relapse or have distant metastases. Thus, the inventors will use retrospective data to check long-term prognosis and if necessary, the inventors will follow-up prospective patients for 5 years. Additionally, since some HPV− HNSCC also show CXCL14 downregulation by promoter methylation, the inventors will expand this study to HPV− HNSCC patients in future. The inventors may use immunofluorescence if there is any limitation in the quantification of IHC.

The foregoing examples of the present disclosure have been presented for purposes of illustration and description. Furthermore, these examples are not intended to limit the disclosure to the form disclosed herein. Consequently, variations and modifications commensurate with the teachings of the description of the disclosure, and the skill or knowledge of the relevant art, are within the scope of the present disclosure. The specific embodiments described in the examples provided herein are intended to further explain the best mode known for practicing the disclosure and to enable others skilled in the art to utilize the disclosure in such, or other, embodiments and with various modifications required by the particular applications or uses of the present disclosure. It is intended that the appended claims be construed to include alternative embodiments to the extent permitted by the prior art.

1. A method of inducing in vivo clearance of a tumor in a subject, the method comprising administering an isolated CXCL14 protein that induces an antitumor immune response in the subject, or a pharmaceutical composition comprising an isolated CXCL14 protein, to the subject in an amount sufficient to reverse immune suppression in the tumor microenvironment thereby inducing the clearance of the tumor from the subject.

2. The method of claim 1, wherein the tumor is a head and neck squamous cell carcinoma (HNSCC), cervical cancer, or anogenital cancer of the vulva, vagina, penis, or anus.

3. The method of claim 1, wherein the tumor has at least a 2-fold reduction in CXCL14 expression compared to a non-tumor tissue.

4. The method of claim 1, wherein the tumor is a human papillomavirus-positive (HPV+) tumor.

5. The method of claim 1, wherein the amount sufficient to reverse immune suppression in the tumor microenvironment is an amount of CXCL14 sufficient to decrease one or more of chemokines CXCL1 and CXCL2.

6. The method of claim 1, further comprising: obtaining a biological sample from the individual, analyzing the biological sample to determine the presence or absence or amount or activity of CXCL14 protein in the sample, and determining whether or not to administer treatment based on the presence, absence, amount or activity of CXCL14 protein in the sample.

7. The method of claim 6, wherein the sample is a saliva sample.

8. The method of claim 1, further comprising: obtaining a biological sample from the individual, analyzing the biological sample to determine CXCL14 mRNA transcript levels in the sample, and determining whether or not to administer treatment based on the CXCL14 mRNA transcript levels in the sample.

9. The method of claim 8, wherein the sample is a saliva sample.

10. The method of claim 1, further comprising: obtaining a biological sample from the individual, analyzing the biological sample to determine CXCL14 gene hypermethylation status in the sample, and determining whether or not to administer treatment based on CXCL14 gene hypermethylation status in the sample.

11. The method of claim 10, wherein the sample is a saliva sample.

12. (canceled)

13. (canceled)

14. A method of treating a subject having an HPV+ tumor comprising adoptive cell transfer of CXCL14-induced CD8+ T and NK cells to the subject.

15. The method of claim 14, wherein the tumor is an HPV+ head and neck squamous cell carcinoma (HNSCC), cervical cancer, or anogenital cancers of the vulva, vagina, penis, or anus.

16. The method of claim 14, further comprising first analyzing a biological sample from the subject to determine a biomarker in the sample selected from the group consisting of:
a. the presence or absence or activity of CXCL14 protein activity;
b. CXCL14 mRNA transcript level; and,
c. CXCL14 gene hypermethylation; and,
determining whether or not to administer the adoptive cell transfer treatment based on the presence, absence or amount or activity of CXCL14 protein activity or CXCL14 mRNA transcript or CXCL14 gene hypermethylation in the sample.

17. The method of claim 16, wherein the biological sample is a saliva sample.

18. The method of claim 16, further comprising adoptive cell transfer of CXCL14-induced CD8+ T and NK cells to the subject if CXCL14 protein level or activity is found to be substantially lower than wild type or a control protein activity level.

19. The method of claim 16, further comprising adoptive cell transfer of CXCL14-induced CD8+ T and NK cells to the subject if the CXCL14 mRNA transcript level in the sample is found to be substantially lower than wild type or a control CXCL14 mRNA level.

20. The method of claim 16, further comprising adoptive cell transfer of CXCL14-induced CD8+ T and NK cells to the subject if a level of CXCL14 gene hypermethylation in the sample is found to be substantially higher than wild type or a control CXCL14 gene hypermethylation level.

21-49. (canceled)

50. A modified CXCL14 protein that induces that induces in vivo clearance of an HPV-positive (HPV+) tumor in a mammal by reversing immune suppression in the tumor microenvironment by decreasing chemokines including at least CXCL1 and/or CXCL2, the protein comprising CXCL14 modified by at least one modification selected from pegylation, acetylation, glycosylation, and covalent linking to an Fc protein.

51-87. (canceled)
